(19) United States
(12) Patent Application Publication
(10) Pub. No.: US 2022/0061710 A1
(43) Pub. Date: Mar. 3, 2022

(54) VIRTUALLY MONITORING GLUCOSE LEVELS IN A PATIENT USING MACHINE LEARNING AND DIGITAL TWIN TECHNOLOGY

(71) Applicant: Twin Health, Inc., Mountain View, CA (US)

(72) Inventors: Frederick Hadley, Sunnyvale, CA (US); James Wilson, Sunnyvale, CA (US); Terrence Chun Yin Poon, Foster City, CA (US); Jahangir Mohammed, Mountain View, CA (US)

(21) Appl. No.: 17/243,470

(22) Filed: Apr. 28, 2021

Related U.S. Application Data
(60) Provisional application No. 63/073,879, filed on Sep. 2, 2020.

(51) Int. Cl. A61B 5/145; A61B 5/00; A61B 5/11; A61B 5/0205; A61B 5/024; G16H 10/40; G16H 40/67; G16H 50/20; G06N 20/00

(52) U.S. Cl. CPC A61B 5/14532; A61B 5/7275; A61B 5/6801; A61B 5/1118; A61B 5/0205; G06N 20/00; A61B 5/742; A61B 5/14546; G16H 10/40; G16H 40/67; G16H 50/20; A61B 5/02438

(57) ABSTRACT

A patient health management platform implements a machine-learned metabolic model to generate a prediction of a patient's glucose level. The platform implements a short-term prediction model to generate a daily prediction of the patient's glucose level based on nutrition data reported by the patient and sensor data and lab test data collected for the patient. The platform implements a long-term prediction model generate a prediction of the patient's glucose level during an extended time period based on sensor data and lab test data collected for the patient. Using the short-term prediction model, the long-term prediction model, or both, the patient health management platform generates predictions of the patient's glucose level and updates a digital twin of the patient's metabolic profile.

### CROSS REFERENCE TO RELATED APPLICATIONS

This application claims the benefit of U.S. Provisional Application No. 63/073,879, filed on Sep. 2, 2020, which is incorporated by reference in its entirety.

### BACKGROUND

### Field of Art

The disclosure relates generally to a patient health management platform, and more specifically, to a personalized treatment platform for virtually monitoring glucose levels in a patient using a machine-learned model and a collection of biosignals.

### Description of the Related Art

Metabolic dysfunction, for example the metabolic dysfunction that occurs in type 2 diabetes, hypertension, lipid problems, heart disease, non-alcoholic fatty liver disease, polycystic ovarian syndrome, cancer, and dementia, is a major contributor to health care costs. Conventional disease management platforms or techniques either ignore or fail to fully understand important markers, such as blood sugar dysregulation, and root causes for these diseases, such as processed foods and a lack of exercise. Traditionally, these platforms are designed to treat symptoms as they arise rather than treating the root cause of the disease—the deterioration of a patient's metabolic health.

Regular monitoring of blood glucose levels is important to both manage and treat certain metabolic diseases, such as diabetes. Conventional platforms often implement blood glucose meters (BGM), which use small blood samples from the finger and test strips, and/or continuous glucose monitors (CGM), which use a sensor inserted into the skin (e.g., on the stomach or arm). However, these technologies do have drawbacks. First, both technologies are invasive and cause patient discomfort. BGMs require a fingerstick (pricking the finger with a lancet to draw blood) for every reading, often multiple times a day. CGMs have a limited life of 10-14 days, and they require re-insertion into the skin for every sensor replacement. Second, both technologies require active participation from patients, which impacts their effectiveness in the long term due to intermittent missed readings, periods of no usage during travel/vacation, etc. Third, both technologies can be costly for patients. BGMs require a new test strip for every reading (recurring monthly cost varies from $30 to over $60 depending on brand and testing frequency), while CGMs require a new sensor every 10-14 days (recurring monthly cost varies from $60 to over $400 depending on brand and model).

### SUMMARY

A patient health management platform for managing a patient's metabolic diseases generates a precision treatment using machine learning techniques and analyzing a unique combination of continuous biosignals (or near continuous or regularly collected biosignals). The platform performs various analyses to establish a personalized metabolic profile for each patient by gaining a deep understanding of how the combination of continuous biosignals impact the patient's metabolic health. These biosignals are input into machine-learned model(s) that recommend personalized treatment based on a unique metabolic profile of the patient. The machine-learned model(s) are trained to predict a patient's response to input biosignals at different stages of his or her treatment. Based on the output of the machine-learned model, the patient health management platform generates personalized recommendations for a patient outlining a treatment plan for improving the patient's metabolic health. To confirm that a patient-specific recommendation effectively addresses a patient's metabolic health, the patient health management evaluates the patient data recorded by a patient to confirm the timeliness, accuracy, and completeness of the recorded data.

One of the machine-learned models implemented by the patient health management platform is a virtual continuous (or near continuous) glucose monitor that is trained to output a prediction of a patient's blood glucose. To generate the virtual continuous glucose monitor, a baseline metabolic model is trained to predict blood glucose on a population-level based on historical data collected from many (e.g., hundreds or thousands) patients. For a particular patient, the baseline metabolic model is trained based on patient-specific biosignals to generate a daily or regular estimation of the patient's blood glucose. In a first embodiment, the virtual continuous glucose monitor generates daily predictions for a patient based on a combination of various inputs, such as lab test data, sensor data, and nutrition data. In a second embodiment where nutrition data is unavailable, the virtual continuous glucose monitor generates a long-term prediction of a patient's blood glucose based on a sensor data and lab test data.

### BRIEF DESCRIPTION OF DRAWINGS

FIG. 1 shows a metabolic health manager for monitoring metabolic health of a patient, performing analytics on the metabolic health data, and providing a patient-specific recommendation for treating metabolic health-related concerns, according to one embodiment.

FIG. 2 is a high-level block illustrating an example of a computing device used in either as a client device, application server, and/or database server, according to one embodiment.

FIG. 3 is a block diagram of the system architecture of a patient health management platform, according to one embodiment.

FIG. 4 is a flowchart illustrating a process for generating a patient-specific recommendation for improving metabolic health of a patient, according to one embodiment.

FIG. 5 is a block diagram of the system architecture of a digital twin module, according to one embodiment.

FIG. 6 is a flowchart illustrating a process for training a machine-learned model to output a representation of a patient's metabolic health, according to one embodiment.

FIG. 7 is an illustration of the process for implementing a machine-learned model to predict a patient-specific metabolic response, according to one embodiment.

FIG. 8A is a block diagram of the system architecture of a glucose twin module, according to one embodiment.

FIG. 8B is a flowchart illustrating a process for determining a short-term prediction of glucose levels for a patient, according to one embodiment.

FIG. 8C is a flowchart illustrating a process for determining a long-term prediction of glucose levels of a patient, according to one embodiment.

FIG. 8D is an illustration of a flowchart for concatenating an A1c prediction generated based on sequential features with an A1c prediction generated based on static features, according to one embodiment.

The figures depict various embodiments of the presented invention for purposes of illustration only. One skilled in the art will readily recognize from the following discussion that alternative embodiments of the structures and methods illustrated herein may be employed without departing from the principles described herein.

### DETAILED DESCRIPTION

### I. System Environment

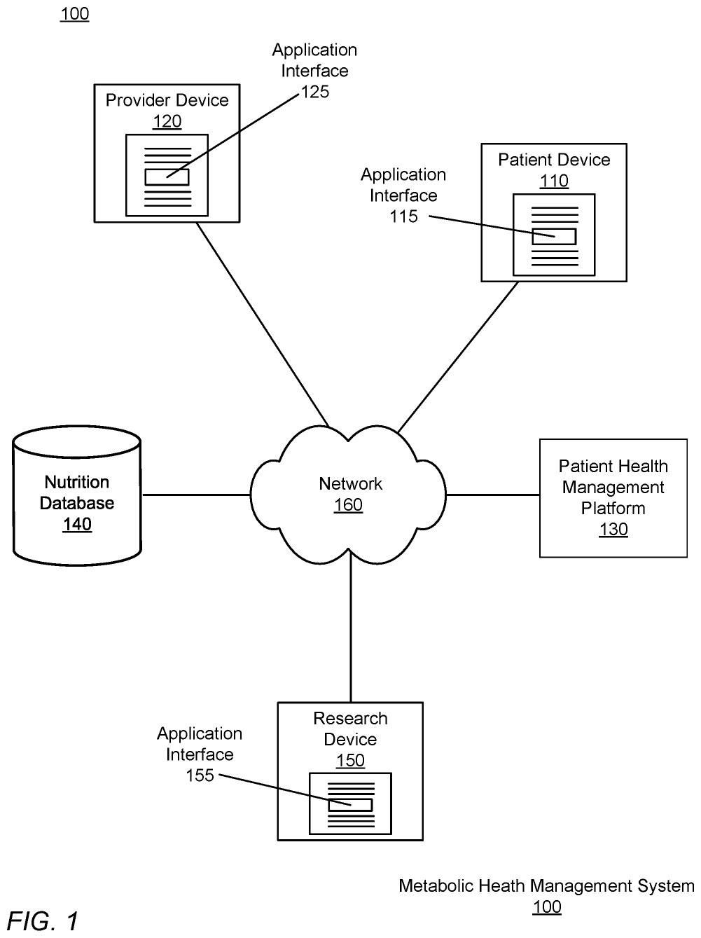

FIG. 1 shows a metabolic health manager 100 for monitoring a patient's metabolic health, for performing analytics on metabolic health data recorded for the patient, and for generating a patient-specific recommendation for treating any metabolic health-related concerns, according to one embodiment. The metabolic health manager 100 includes patient device(s) 110, provider device(s) 120, a patient health management platform 130, a nutrition database 140, research device(s) 150 and a network 160. However, in other embodiments, the system 100 may include different and/or additional components. For example, the patient device 110 can represent thousands or millions of devices for patients (e.g., patient mobile devices) that interact with the system in locations around the world. Similarly, the provider device 120 can represent thousands or millions of devices of providers (e.g., mobile phones, laptop computers, in-provider-office recording devices, etc.). In some cases, a single provider may have more than one device that interacts with the platform 130.

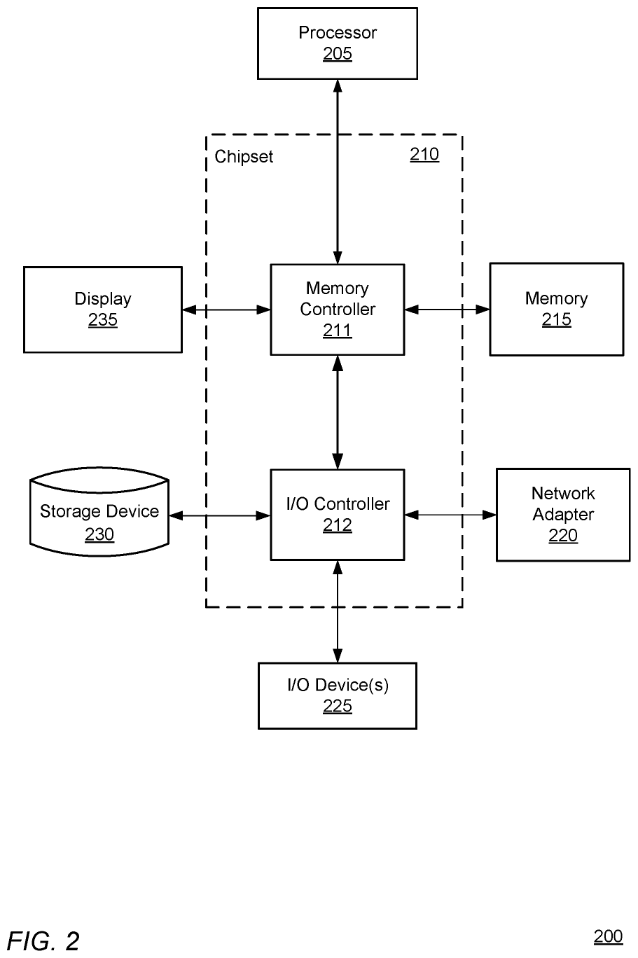

The patient device 110 is a computing device with data processing and data communication capabilities that is capable of receiving inputs from a patient. An example physical implementation is described more completely below with respect to FIG. 2. In addition to data processing, the patient device 110 may include functionality that allows the device 110 to record speech responses articulated by a patient operating the device (e.g., a microphone), and to graphically present data to a patient (e.g., a graphics display). Examples of the patient device 110 include desktop computers, laptop computers, portable computers, GOOGLE HOME, AMAZON ECHO, etc. The patient device 110 may present information generated by the communication platform 130 via a mobile application configured to display and record patient responses. For example, through a software application interface 115, a patient may receive a recommendation or an update regarding their metabolic health.

Application 115 provides a user interface (herein referred to as a “patient dashboard”) that is displayed on a screen of the patient device 110 and allows a patient to input commands to control the operation of the application 115. The patient dashboard enables patients to track and manage changes in a patient's metabolic health. For example, the dashboard allows patients to observe changes in their metabolic health over time, receive recommendation notifications, exchange messages about treatment with a health care provider, and so on. The application 115 may be coded as a web page, series of web pages, or content otherwise coded to render within an internet browser. The application 115 may also be coded as a proprietary application configured to operate on the native operating system of the patient device 110. In addition to providing the dashboard, application 115 may also perform some data processing on biological and food data locally using the resources of patient device 110 before sending the processed data through the network 150. Patient data sent through the network 150 is received by the patient health management platform 130 where it is analyzed and processed for storage and retrieval in conjunction with a database.

Similarly, a provider device 120 is a computing device with data processing and data communication capabilities that is capable of receiving input from a provider. The provider device 120 is configured to present a patient's medical history or medically relevant data (i.e., a display screen). The above description of the functionality of the patient device 110 also can apply to the provider device 120. The provider device 120 can be a personal device (e.g., phone, tablet) of the provider, a medical institution computer (e.g., a desktop computer of a hospital or medical facility), etc. In addition, the provider device 120 can include a device that sits within the provider office such that the patient can interact with the device inside the office. In such implementations, the provider device is a customized device with audio and/or video capabilities (e.g., a microphone for recording, a display screen for text and/or video, an interactive user interface, a network interface, etc.). The provider device 120 may also present information to medical providers or healthcare organizations via a mobile application similar to the application described with reference to patient device 110.

Application 125 provides a user interface (herein referred to as a “provider dashboard”) that is displayed on a screen of the provider device 120 and allows a medical provider or trained professional/coach to input commands to control the operation of the application 125. The provider dashboard enables providers to track and manage changes in a patient's metabolic health. The application 125 may be coded as a web page, series of web pages, or content otherwise coded to render within an internet browser. The application 125 may also be coded as a proprietary application configured to operate on the native operating system of the patient device 110.

The patient health management platform 130 is a medium for dynamically generating recommendations for improving a patient's metabolic health based on biological data recorded from a plurality of sources including wearable sensors (or other types of IoT sensors), lab tests, etc., and food or diet-related data recorded by the patient. The patient health management platform 130 predicts a patient's metabolic response based on periodically recorded patient data (e.g., nutrition data, symptom data, lifestyle data). Accordingly, a patient's metabolic response describes a change in metabolic health for a patient resulting from the food they most recently consumed and their current metabolic health. Based on such a change, the platform 130 generates a recommendation including instructions for a patient to improve their metabolic health or to maintain their improved metabolic health. Additionally, in real-time or near real-time, the patient health management platform 130 may provide feedback to a patient identifying potential inconsistencies or errors in the food or biological data entered manually by the patient based on a comparison of the patient's true metabolic state and their predicted metabolic state.

The nutrition database 140 stores nutrition data extracted from a collection of nutrient sources, for example food or vitamins. Data within the nutrition database 140 may be populated using data recorded by a combination of public sources and third-party entities such as the USDA, research programs, or affiliated restaurants. The stored data may include, but is not limited to, nutrition information (for example, calories, macromolecule measurements, vitamin concentrations, cholesterol measurements, or other facts) for individual foods or types of foods and relationships between foods and metabolic responses (for example, an impact of a given food on insulin sensitivity). Data stored in the nutrition database 140 may be applicable to an entire population (i.e., general nutrition information) or personalized to an individual patient (i.e., a personalized layer of the nutrition database). For example, the nutrition database 140 may store information describing a patient's particular biological (i.e., metabolic) response to a food. In such embodiments, the nutrition database 140 may be updated based on feedback from the patient health management platform 140.

Application 155 provides a user interface (herein referred to as a “research dashboard”) that is displayed on a screen of the research device 150 and allows a researcher to input commands to control the operation of the application 155. The research dashboard enables providers to track and manage changes in a patient's metabolic health. The application 155 may be coded as a web page, series of web pages, or content otherwise coded to render within an internet browser. The application 155 may also be coded as a proprietary application configured to operate on the native operating system of the patient device 110.

Interactions between the patient device 110, the provider device 120, the patient health management platform 130, and the nutrition database 140 are typically performed via the network 150, which enables communication between the patient device 120, the provider device 130, and the patient communication platform 130. In one embodiment, the network 150 uses standard communication technologies and/or protocols including, but not limited to, links using technologies such as Ethernet, 802.11, worldwide interoperability for microwave access (WiMAX), 3G, 4G, LTE, digital subscriber line (DSL), asynchronous transfer mode (ATM), InfiniBand, and PCI Express Advanced Switching. The network 150 may also utilize dedicated, custom, or private communication links. The network 150 may comprise any combination of local area and/or wide area networks, using both wired and wireless communication systems.

FIG. 2 is a high-level block diagram illustrating physical components of an example computer 200 that may be used as part of a client device (e.g., devices 110, 120, 150), application server 130, and/or database server 140 from FIG. 1, according to one embodiment. Illustrated is a chipset 210 coupled to at least one processor 205. Coupled to the chipset 210 is volatile memory 215, a network adapter 220, an input/output (I/O) device(s) 225, a storage device 230 representing a non-volatile memory, and a display 235. In one embodiment, the functionality of the chipset 210 is provided by a memory controller 211 and an I/O controller 212. In another embodiment, the memory 215 is coupled directly to the processor 205 instead of the chipset 210. In some embodiments, memory 215 includes high-speed random access memory (RAM), such as DRAM, SRAM, DDR RAM or other random access solid state memory devices.

The storage device 230 is any non-transitory computer-readable storage medium, such as a hard drive, compact disk read-only memory (CD-ROM), DVD, or a solid-state memory device. The memory 215 holds instructions and data used by the processor 205. The I/O device 225 may be a touch input surface (capacitive or otherwise), a mouse, track ball, or other type of pointing device, a keyboard, or another form of input device. The display 235 displays images and other information for the computer 200. The network adapter 220 couples the computer 200 to the network 150.

As is known in the art, a computer 200 can have different and/or other components than those shown in FIG. 2. In addition, the computer 200 can lack certain illustrated components. In one embodiment, a computer 200 acting as server 140 may lack a dedicated I/O device 225, and/or display 218. Moreover, the storage device 230 can be local and/or remote from the computer 200 (such as embodied within a storage area network (SAN)), and, in one embodiment, the storage device 230 is not a CD-ROM device or a DVD device.

Generally, the exact physical components used in a client device 110 will vary in size, power requirements, and performance from those used in the application server 130 and the database server 140. For example, client devices 110, which will often be home computers, tablet computers, laptop computers, or smart phones, will include relatively small storage capacities and processing power, but will include input devices and displays. These components are suitable for user input of data and receipt, display, and interaction with notifications provided by the application server 130. In contrast, the application server 130 may include many physically separate, locally networked computers each having a significant amount of processing power for carrying out the asthma risk analyses introduced above. In one embodiment, the processing power of the application server 130 provided by a service such as Amazon Web Services™. Also, in contrast, the database server 140 may include many, physically separate computers each having a significant amount of persistent storage capacity for storing the data associated with the application server.

As is known in the art, the computer 200 is adapted to execute computer program modules for providing functionality described herein. A module can be implemented in hardware, firmware, and/or software. In one embodiment, program modules are stored on the storage device 230, loaded into the memory 215, and executed by the processor 205.

### II. Overview of Metabolic Health Manager

In the United States, treating non-communicable diseases including, but not limited to, diabetes, hyper-tension, high-cholesterol, heart disease, obesity, fatty liver disease, arthritis, irritable bowel syndrome (IBS), and infertility, is a multi-billion-dollar industry. Still, these diseases account for over 2 million deaths annually. Conventional treatments are directed towards addressing and alleviating symptoms of each disease, but they fail to recognize that the root of all the aforementioned diseases is an impaired metabolism. By addressing root cause metabolic impairments, a patient's disease may not just be managed on a per symptom basis, but it may be reversed entirely. Accordingly, a treatment or system for generating a treatment directed towards treating metabolic impairments in patients suffering from such diseases could be more effective and most cost-efficient. Because the patient health management platform 100 aims to treat a patient's metabolic impairments, a patient using the patient health management platform 100 for an extended time period may transition from a first state of impaired metabolism to a second state of functional metabolism to a third state of optimal metabolism.

The patient health management platform 130, as described herein, recognizes that a patient's body is a unique system in a unique state in which metabolism is a core biochemical process. Accordingly, the treatment and nutrition recommendations generated by the platform 130 are tailored to suit a patient's unique metabolic state and the unique parameters or conditions that impact or have previously impacted their metabolic state. To enable a patient to achieve good or optimal metabolic health, the platform 130 records measurements of various factors and aims to improve these measurements to levels representative of an optimized metabolic state. For example, five factors commonly considered include blood sugar, triglycerides, good cholesterol (high-density lipoprotein), blood pressure, and waist circumference. Each human body is different and continuously evolving. To guide a patient towards optimal metabolic health, the platform establishes a deep understanding of the dynamic states of each human body over time by capturing continuous biosignals and deriving insights from these biosignals.

For each patient, the platform 130 leverages a combination of personalized treatments that are tailored to a patient's unique metabolic state based on a combination of timely, accurate, and complete recordings of metabolic biosignals. Such measurements are collectively referred to herein as “TAC measurements.” The platform determines a current metabolic state of a human body by analyzing a unique combination of continuous biosignals received from various sources including, but not limited to, near-real-time data from wearable sensors (e.g. continuous blood glucose, heart rate, etc.), periodic lab tests (e.g., blood work), nutrition data (e.g., macronutrients, micronutrients, and biota nutrients from food and supplements of the patient), medicine data (e.g., precise dosage and time of medications taken by the patient), and symptom data (e.g., headache, cramps, frequent urination, mood, energy, etc., reported by each patient via a mobile app). This analysis is performed continuously to establish a time series of metabolic states. As a result, the platform understands not only the current state of each patient, but also the full history of states that led to the current state. Using a patient's current metabolic state and their full history of metabolic states, the platform is able to deeply personalize the treatment for each patient.

The platform applies various technologies and processing techniques to gain a deep understanding of the combination of factors contributing to a patient's metabolic state and to establish a personalized metabolic profile for each patient. For example, the platform implements a combination of analytics (e.g., analyzing trends, outliers, and anomalies in biosignals as well as correlations across multiple biosignals), rule based artificial intelligence (AI), machine learning-based AI, and automated cohorting or clustering.

For the sake of explanation, the concepts and techniques described herein are described with reference to diabetes. However, one of skill in the art would recognize that the concepts and techniques may also be applied to any other disease resulting from an impaired metabolism. As will be described herein, a patient's metabolic health describes the overall effectiveness of their metabolism. For example, a patient's metabolic health may be categorized as impaired, functional, or optimal. To gain insight into a patient's metabolic health, the patient health management platform 130 identifies metabolic states occurring over a time period and changes between those metabolic states. As described herein, a metabolic state represents a patient's state of metabolic health at a specific time (e.g., a state of metabolic health resulting from consumption of a particular food or adherence to a particular medication/treatment).

In addition, the term “continuously” is used throughout the description to characterize the collection of biosignals and other data regarding the patient. This term can refer to a rate of collection that is truly continuous (e.g., a constantly recorded value) or near continuous (e.g., collection at every time point or time increment, such as every millisecond, second, or minute), such as biosignals recorded by a wearable device. In some cases, continuously recorded data may refer to particular biosignals that occur semi-regularly, such as a lab test that is taken at a recurring time interval (e.g., every 10 minutes, 30 minutes, hour, 5 hours, day or number of days, week or number of weeks, etc.). The term “continuously” does not exclude situations in which wearable sensors may be removed during certain activities or at times of day (e.g., while showering). In other embodiments, the platform collects multiple biosignals that, in combination, represent a continuous or near continuous signal collection even though some biosignals are collected more frequently than others.

### III. Biosignal Data

A patient health management platform receives biosignal data for a patient from a variety of sources including, but not limited to, wearable sensor data, lab test data, nutrition data, medication data, symptom data.

A patient using the metabolic health manager is outfitted with one or more wearable sensors configured to continuously record biosignals, herein referred to as wearable sensor data. Wearable sensor data includes, but is not limited to, biosignals describing a patient's heart rate, record of exercise (e.g., steps, average number of active minutes), quality of sleep (e.g., sleep duration, sleep stages), a blood glucose measurement, a ketone measurement, systolic and diastolic blood pressure measurements, weight, BMI, percentage of fat, percentage of muscle, bone mass measurement, and percent composition of water. A wearable sensor may be a sensor that is periodically removable by a patient (e.g., a piece of jewelry worn in contact with a patient's skin to record such biosignals) or a non-removable device/sensor embedded into a patient's skin (e.g., a glucose patch). Whenever worn or activated to record wearable sensor data, the sensor continuously records one or more of the measurements listed above. In some implementations, a wearable sensor may record different types of wearable sensor data at different rates or intervals. For example, the wearable sensor may record blood glucose measurements, heart rate measurements, and steps in 15 second intervals, but record blood pressure measurements, weight measurements, and sleep trends in daily intervals.

The patient health management platform also receives lab test data recorded for a patient. As described herein, lab test data describes the results of lab tests performed on the patient. Examples of lab test data include, but are not limited to, blood tests or blood draw analysis. Compared to the frequencies at which wearable sensor data is recorded, lab test data may be recorded at longer intervals, for example bi-weekly or monthly. In some implementations, the patient health management platform receives data measured from 126-variable blood tests.

The patient health management platform may also receive nutrition data 320 describing food that a patient is consuming or has consumed. Via an interface (e.g., the application interface) presented on the patient device, a patient enters a record of food that they have consumed on a per meal basis and a time at which each item of food was consumed. Alternatively, the patient may enter the record for food on a daily basis. The patient health management platform extracts nutrition details (e.g., macronutrient, micronutrient, and biota nutrient data) from a nutrition database (not shown) based on the food record entered by the patient. As an example, via a patient device, a patient may record that they consumed two bananas for breakfast at 7:30 AM. The record of the two bananas is communicated to the patient health management platform 350 and the patient health management platform accesses, from a nutrition database, nutrient data including the amount of potassium in a single banana. The accessed nutrient data is returned to the patient health management platform as an update to the recorded nutrition data. Via the same interface or one similar to the interface used to record food consumed, a patient may record and communicate medication data and symptom data to the patient health management platform. Medication data describes a type of medication taken, a time at which the medication was taken, and an amount of the medication taken. In addition to nutrition data and medication data, the patient health management platform may receive descriptions of a patient's energy, mood, or general level of satisfaction with their lifestyle, treatment plan, and disease management.

Examples of biosignal data recorded and communicated to the patient health management platform include, but are not limited to, those listed in Table 1. Table 1 also lists a source for recording each example of biosignal data.

TABLE 1
Example Biosignal Data and Source

| Category | Type | Signal | Source |
|---|---|---|---|
| Sensor Data | Biomarker | Weight | Body Composition Scale |
| | Biomarker | Body fat % | Body Composition Scale |
| | Biomarker | Subcutaneous fat % | Body Composition Scale |
| | Biomarker | Visceral fat % | Body Composition Scale |
| | Biomarker | Body water % | Body Composition Scale |
| | Biomarker | Muscle % | Body Composition Scale |
| | Biomarker | Bone mass | Body Composition Scale |
| | Biomarker | Basal metabolic rate | Body Composition Scale |
| | Biomarker | Protein | Body Composition Scale |
| | Biomarker | Lean body weight | Body Composition Scale |
| | Biomarker | Muscle mass | Body Composition Scale |
| | Biomarker | Metabolic age | Body Composition Scale |
| | Biomarker | Continuous Blood | Continuous Glucose Meter |
| | | Glucose | |
| | Biomarker | Ketones | Ketone Meter |
| | Biomarker | Systolic BP | Blood Pressure Meter |
| | Biomarker | Diastolic BP | Blood Pressure Meter |
| | Heart | Resting Heart Rate | Fitness Watch |
| | Heart | Continuous Heart | Fitness Watch |
| | | Rate | |
| Lab Test Data | Biomarker | Skin Temperature | Patient Investigation/Test |
| | Biomarker | Oxygen Saturation | Patient Investigation/Test |
| | Biomarker | Waist Circumference | Patient Investigation/Test |
| | Biomarker | Age | Patient Interview |
| | Biomarker | Gender | Patient Interview |
| | Biomarker | Height | Patient Interview |
| | Biomarker | BMI | Patient Interview |
| | Biomarker | HbA1c | Blood Test |
| | Biomarker | 5dg-cgm | Blood Test |
| | Biomarker | 1dg-cgm | Blood Test |
| | Biomarker | Insulin | Blood Test |
| | Biomarker | Fructosamine | Blood Test |
| | Biomarker | C-Peptide | Blood Test |
| | Biomarker | HOMA-IR | Blood Test |
| | Biomarker | 5dk | Blood Test |
| | Biomarker | Cholesterol | Blood Test |
| | Biomarker | Triglycerides | Blood Test |
| | Biomarker | HDL Cholesterol | Blood Test |
| | Biomarker | LDL Cholesterol | Blood Test |
| | Biomarker | VLDL Cholesterol | Blood Test |
| | Biomarker | Triglyceride/HDL | Blood Test |
| | | Ratio | |
| | Biomarker | Total Cholesterol/ | Blood Test |
| | | HDL Ratio | |
| | Biomarker | Non - HDL | Blood Test |
| | | Cholesterol | |
| | Biomarker | LDL/HDL Ratio | Blood Test |
| | Biomarker | Total Iron Binding | Blood Test |
| | | Capacity (TIBC) | |
| | Biomarker | Serum Iron | Blood Test |
| | Biomarker | % Transferrin | Blood Test |
| | | Saturation | |
| | Biomarker | Amylase | Blood Test |
| | Biomarker | Lipase | Blood Test |
| | Biomarker | Ferritin | Blood Test |
| | Biomarker | Homocysteine | Blood Test |
| | Biomarker | Magnesium | Blood Test |
| | Biomarker | ALT | Blood Test |
| | Biomarker | AST | Blood Test |
| | Biomarker | ALP | Blood Test |
| | Biomarker | Total Bilirubin | Blood Test |
| | Biomarker | Direct Bilirubin | Blood Test |
| | Biomarker | Indirect Bilirubin | Blood Test |
| | Biomarker | Gamma Glutamyl | Blood Test |
| | | Transferase (GGT) | |
| | Biomarker | Protein | Blood Test |
| | Biomarker | Albumin | Blood Test |
| | Biomarker | A/G Ratio | Blood Test |
| | Biomarker | Globulin | Blood Test |
| | Biomarker | Urea | Blood Test |
| | Biomarker | Creatinine | Blood Test |
| | Biomarker | Uric Acid | Blood Test |
| | Biomarker | GFR | Blood Test |
| | Biomarker | Blood urea nitrogen | Blood Test |
| | | (BUN) | |
| | Biomarker | BUN/Creatinine | Blood Test |
| | | Ratio | |
| | Biomarker | Lipoprotein(a) | Blood Test |
| | Biomarker | Apolipoprotein A1 | Blood Test |
| | Biomarker | ApoB | Blood Test |
| | Biomarker | hs-CRP | Blood Test |
| | Biomarker | Apo B/Apo A1 | Blood Test |
| | | Ratio | |
| | Biomarker | LP-PLA2 | Blood Test |
| | Biomarker | Total Triiodothyronine | Blood Test |
| | | [T3] | |
| | Biomarker | Total Thyroxine [T4] | Blood Test |
| | Biomarker | TSH | Blood Test |
| | Biomarker | Sodium | Blood Test |
| | Biomarker | Chloride | Blood Test |
| | Biomarker | Potassium | Blood Test |
| | Biomarker | Bicarbonate | Blood Test |
| | Biomarker | Calcium | Blood Test |
| | Biomarker | Phosphorous | Blood Test |
| | Biomarker | Anion Gap | Blood Test |
| | Biomarker | Vitamin A | Blood Test |
| | Biomarker | Vitamin D2 | Blood Test |
| | Biomarker | Vitamin D3 | Blood Test |
| | Biomarker | Vitamin D Total | Blood Test |
| | Biomarker | Vitamin E | Blood Test |
| | Biomarker | Vitamin K | Blood Test |
| | Biomarker | Vitamin B1/Thiamin | Blood Test |
| | Biomarker | Vitamin B2/ | Blood Test |
| | | Riboflavin | |
| | Biomarker | Vitamin B3/ | Blood Test |
| | | Nicotinic Acid | |
| | Biomarker | Vitamin B5/ | Blood Test |
| | | Pantothenic Acid | |
| | Biomarker | Vitamin B6/ | Blood Test |
| | | Pyridoxal-5- | |
| | | Phosphate | |
| | Biomarker | Vitamin B7/Biotin | Blood Test |
| | Biomarker | Vitamin B9/Folic | Blood Test |
| | | Acid | |
| | Biomarker | Vitamin B12/ | Blood Test |
| | | Cobalamin | |
| | Biomarker | Cortisol | Blood Test |
| | Biomarker | Cystatin C | Blood Test |
| | Biomarker | Serum Zinc | Blood Test |
| | Biomarker | Serum Copper | Blood Test |
| | Biomarker | Basophils - | Blood Test |
| | | Absolute Count | |
| | Biomarker | Eosinophils - | Blood Test |
| | | Absolute Count | |
| | Biomarker | Lymphocytes - | Blood Test |
| | | Absolute Count | |
| | Biomarker | Monocytes - | Blood Test |
| | | Absolute Count | |
| | Biomarker | Mixed - | Blood Test |
| | | Absolute Count | |
| | Biomarker | Neutrophils - | Blood Test |
| | | Absolute Count | |
| | Biomarker | Basophils | Blood Test |
| | Biomarker | Eosinophils | Blood Test |
| | Biomarker | Immature | Blood Test |
| | | Granulocytes (Ig) | |
| | Biomarker | Immature | Blood Test |
| | | Granulocyte | |
| | | Percentage (Ig %) | |
| | Biomarker | White Blood Cells | Blood Test |
| | | (Leucocytes Count) | |
| | Biomarker | Lymphocyte | Blood Test |
| | | Percentage | |
| | Biomarker | Mean Corpuscular | Blood Test |
| | | Hemoglobin (Mch) | |
| | Biomarker | Mean Corp. Hemo. | Blood Test |
| | | Conc. (Mchc) | |
| | Biomarker | MCV | Blood Test |
| | Biomarker | Monocytes | Blood Test |
| | Biomarker | Mean Platelet | Blood Test |
| | | Volume (Mpv) | |
| | Biomarker | Neutrophils | Blood Test |
| | Biomarker | Nucleated Red Blood | Blood Test |
| | | Cells | |
| | Biomarker | Nucleated Red Blood | Blood Test |
| | | Cells % | |
| | Biomarker | Plateletcrit (Pct) | Blood Test |
| | Biomarker | Hematocrit | Blood Test |
| | Biomarker | Platelet Distribution | Blood Test |
| | | Width (Pdw- SD) | |
| | Biomarker | Platelet To Large Cell | Blood Test |
| | | Ratio (Plcr) | |
| | Biomarker | Platelet Count | Blood Test |
| | Biomarker | Red Blood Cell Count | Blood Test |
| | Biomarker | Red Cell Distribution | Blood Test |
| | | Width (Rdw-Cv) | |
| | Biomarker | Red Cell Distribution | Blood Test |
| | | Width - Sd (Rdw-Sd) | |
| | Biomarker | Blood pH | Blood Test |
| | Biomarker | Hemoglobin | Blood Test |
| | Biomarker | ACCP | Blood Test |
| | Biomarker | ANA | Blood Test |
| | Biomarker | Cadmium | Blood Test |
| | Biomarker | Cobalt | Blood Test |
| | Biomarker | Chromium | Blood Test |
| | Biomarker | Caesium | Blood Test |
| | Biomarker | Mercury | Blood Test |
| | Biomarker | Manganese | Blood Test |
| | Biomarker | Molybdenum | Blood Test |
| | Biomarker | Nickel | Blood Test |
| | Biomarker | Lead | Blood Test |
| | Biomarker | Antimony | Blood Test |
| | Biomarker | Selenium | Blood Test |
| | Biomarker | Tin | Blood Test |
| | Biomarker | Strontium | Blood Test |
| | Biomarker | Thallium | Blood Test |
| | Biomarker | Uranium | Blood Test |
| | Biomarker | Vanadium | Blood Test |
| | Biomarker | Silver | Blood Test |
| | Biomarker | Aluminium | Blood Test |
| | Biomarker | Arsenic | Blood Test |
| | Biomarker | Barium | Blood Test |
| | Biomarker | Beryllium | Blood Test |
| | Biomarker | Bismuth | Blood Test |
| | Biomarker | Testosterone | Blood Test |
| Lifestyle Data | Sleep | Sleep Quality | Fitness Watch |
| | Sleep | Minutes Asleep | Fitness Watch |
| | Sleep | Minutes Awake | Fitness Watch |
| | Sleep | Minutes Light Sleep | Fitness Watch |
| | Sleep | Minutes Deep Sleep | Fitness Watch |
| | Sleep | Minutes REM Sleep | Fitness Watch |
| | Exercise | Activity Calories | Fitness Watch |
| | Exercise | Marginal Calories | Fitness Watch |
| | Exercise | BMR Calories | Fitness Watch |
| | Exercise | Total Calories Burned | Fitness Watch |
| | Exercise | Continuous Steps (per | Fitness Watch |
| | | minute) | |
| | Exercise | Fairly Active Minutes | Fitness Watch |
| | Exercise | Light Active Minutes | Fitness Watch |
| | Exercise | Very Active Minutes | Fitness Watch |
| | Exercise | Sedentary Minutes | Fitness Watch |
| | Exercise | Stress | Fitness Watch |
| | Patient | Age | Patient Interview |
| | Information | | |
| | Patient | Gender | Patient Interview |
| | Information | | |
| | Patient | Height | Patient Interview |
| | Information | | |
| | Patient | BMI | Patient Interview |
| | Information | | |
| | Patient | Vegetarian | Patient Interview |
| | Information | | |
| | Patient | Tobacco | Patient Interview |
| | Information | | |
| | Patient | Alcohol | Patient Interview |
| | Information | | |
| | Patient | Caffeine | Patient Interview |
| | Information | | |
| | Family | Father Diabetic? | Patient Interview |
| | Information | | |
| | Family | Mother Diabetic? | Patient Interview |
| | Information | | |
| | Family | Sibling Diabetic? | Patient Interview |
| | Information | | |
| | Family | Grandparents Diabetic? | Patient Interview |
| | Information | | |
| | Happiness | Energy | Patient Health Management App |
| | Happiness | Mood | Patient Health Management App |
| | Happiness | Cuisine Preferences | Patient Health Management App |
| | Happiness | Food Ratings | Patient Health Management App |
| | Happiness | Meal Ratings | Patient Health Management App |
| | Happiness | Exercise Preferences | Patient Health Management App |
| Symptom Data | Symptom | Headache | Patient Health Management App |
| | Symptom | Cramps | Patient Health Management App |
| | Symptom | Numbness | Patient Health Management App |
| | Symptom | Frequent Urination | Patient Health Management App |
| | Symptom | Blurred Vision | Patient Health Management App |
| | Symptom | Tiredness | Patient Health Management App |
| | Symptom | Excess hunger | Patient Health Management App |
| | Symptom | Giddiness | Patient Health Management App |
| | Symptom | Nausea | Patient Health Management App |
| | Symptom | Vomiting | Patient Health Management App |
| | Symptom | Diarrhea | Patient Health Management App |
| | Symptom | Excess thirst | Patient Health Management App |
| | Symptom | Constipation | Patient Health Management App |
| | Symptom | Erectile dysfunction | Patient Health Management App |
| | Symptom | Sleeplessness | Patient Health Management App |
| Medication Data | Medication | Diabetes Medicine | Patient Health Management App |
| | Medication | Insulin | Patient Health Management App |
| | Medication | Hypertension | Patient Health Management App |
| | | Medicines | |
| | Medication | Cholesterol | Patient Health Management App |
| | | Medicines | |
| | Medication | Obesity Medicines | Patient Health Management App |
| | Medication | Heart Medicines | Patient Health Management App |
| | Medication | Arthritis Medicines | Patient Health Management App |
| Nutrition Data | Macronutrients | Net Carb | Nutrition Database/Patient |
| | | | Health Management App |
| | Macronutrients | Calories consumed | Nutrition Database/Patient |
| | | | Health Management App |
| | Macronutrients | Net GI Carb | Nutrition Database/Patient |
| | | | Health Management App |
| | Macronutrients | Fiber | Nutrition Database/Patient |
| | | | Health Management App |
| | Macronutrients | Fat | Nutrition Database/Patient |
| | | | Health Management App |
| | Macronutrients | Protein | Nutrition Database/Patient |
| | | | Health Management App |
| | Macronutrients | Total Carb | Nutrition Database/Patient |
| | | | Health Management App |
| | Micronutrients | Fructose | Nutrition Database/Patient |
| | | | Health Management App |
| | Micronutrients | Sodium | Nutrition Database/Patient |
| | | | Health Management App |
| | Micronutrients | Potassium | Nutrition Database/Patient |
| | | | Health Management App |
| | Micronutrients | Magnesium | Nutrition Database/Patient |
| | | | Health Management App |
| | Micronutrients | Calcium | Nutrition Database/Patient |
| | | | Health Management App |
| | Micronutrients | Chromium | Nutrition Database/Patient |
| | | | Health Management App |
| | Micronutrients | Omega 3 | Nutrition Database/Patient |
| | | | Health Management App |
| | Micronutrients | Omega 6 | Nutrition Database/Patient |
| | | | Health Management App |
| | Micronutrients | ALA | Nutrition Database/Patient |
| | | | Health Management App |
| | Micronutrients | Q10 | Nutrition Database/Patient |
| | | | Health Management App |
| | Micronutrients | Biotin | Nutrition Database/Patient |
| | | | Health Management App |
| | Micronutrients | Flavonoids | Nutrition Database/Patient |
| | | | Health Management App |
| | Glycemic | Improve IS | Nutrition Database/Patient |
| | Controllers | | Health Management App |
| | Glycemic | Inhibit GNG | Nutrition Database/Patient |
| | Controllers | | Health Management App |
| | Glycemic | Inhibit Carb | Nutrition Database/Patient |
| | Controllers | Absorption | Health Management App |
| | Glycemic | Improve Insulin | Nutrition Database/Patient |
| | Controllers | Secretion | Health Management App |
| | Glycemic | Impr B-Cell Regen | Nutrition Database/Patient |
| | Controllers | | Health Management App |
| | Glycemic | Inhibit Hunger | Nutrition Database/Patient |
| | Controllers | | Health Management App |
| | Glycemic | Inhibit Glucose | Nutrition Database/Patient |
| | Controllers | Kidney Reabsorption | Health Management App |
| | Biotanutrients | Lactococcus sp. | Nutrition Database/Patient |
| | | | Health Management App |
| | Biotanutrients | Lactobacillus sp. | Nutrition Database/Patient |
| | | | Health Management App |
| | Biotanutrients | Leuconostoc sp. | Nutrition Database/Patient |
| | | | Health Management App |
| | Biotanutrients | Streptococcus sp. | Nutrition Database/Patient |
| | | | Health Management App |
| | Biotanutrients | Bifidobacterium sp. | Nutrition Database/Patient |
| | | | Health Management App |
| | Biotanutrients | Saccharomyces sp. | Nutrition Database/Patient |
| | | | Health Management App |
| | Biotanutrients | Bacillus sp. | Nutrition Database/Patient |
| | | | Health Management App |
| | Glycemic | Glycemic Index | Nutrition Database/Patient |
| | Impact | | Health Management App |
| | Fats | Saturated fat | Nutrition Database/Patient |
| | | | Health Management App |
| | Fats | Monounsaturated fat | Nutrition Database/Patient |
| | | | Health Management App |
| | Fats | Polyunsaturated fat | Nutrition Database/Patient |
| | | | Health Management App |
| | Fats | Trans fat | Nutrition Database/Patient |
| | | | Health Management App |
| | Fats | Cholesterol | Nutrition Database/Patient |
| | | | Health Management App |
| | Proteins | Histidine | Nutrition Database/Patient |
| | | | Health Management App |
| | Proteins | Isoleucine | Nutrition Database/Patient |
| | | | Health Management App |
| | Proteins | Lysine | Nutrition Database/Patient |
| | | | Health Management App |
| | Proteins | Methionine + | Nutrition Database/Patient |
| | | Cysteine | Health Management App |
| | Proteins | Phenylalanine + | Nutrition Database/Patient |
| | | Tyrosine | Health Management App |
| | Proteins | Tryptophan | Nutrition Database/Patient |
| | | | Health Management App |
| | Proteins | Threonine | Nutrition Database/Patient |
| | | | Health Management App |
| | Proteins | Valine | Nutrition Database/Patient |
| | | | Health Management App |
| | Vitamins/Minerals | Vitamin A | Nutrition Database/Patient |
| | | | Health Management App |
| | Vitamins/Minerals | Vitamin C | Nutrition Database/Patient |
| | | | Health Management App |
| | Vitamins/Minerals | Vitamin D | Nutrition Database/Patient |
| | | | Health Management App |
| | Vitamins/Minerals | Vitamin E | Nutrition Database/Patient |
| | | | Health Management App |
| | Vitamins/Minerals | Vitamin K | Nutrition Database/Patient |
| | | | Health Management App |
| | Vitamins/Minerals | B1 | Nutrition Database/Patient |
| | | | Health Management App |
| | Vitamins/Minerals | B12 | Nutrition Database/Patient |
| | | | Health Management App |
| | Vitamins/Minerals | B2 | Nutrition Database/Patient |
| | | | Health Management App |
| | Vitamins/Minerals | B3 | Nutrition Database/Patient |
| | | | Health Management App |
| | Vitamins/Minerals | B5 | Nutrition Database/Patient |
| | | | Health Management App |
| | Vitamins/Minerals | B6 | Nutrition Database/Patient |
| | | | Health Management App |
| | Vitamins/Minerals | Folate | Nutrition Database/Patient |
| | | | Health Management App |
| | Vitamins/Minerals | Copper | Nutrition Database/Patient |
| | | | Health Management App |
| | Vitamins/Minerals | Iron | Nutrition Database/Patient |
| | | | Health Management App |
| | Vitamins/Minerals | Zinc | Nutrition Database/Patient |
| | | | Health Management App |
| | Vitamins/Minerals | Manganese | Nutrition Database/Patient |
| | | | Health Management App |
| | Vitamins/Minerals | Phosphorus | Nutrition Database/Patient |
| | | | Health Management App |
| | Vitamins/Minerals | Selenium | Nutrition Database/Patient |
| | | | Health Management App |
| | Vitamins/Minerals | Omega 6/omega 3 | Nutrition Database/Patient |
| | | | Health Management App |
| | Vitamins/Minerals | Zinc/Copper | Nutrition Database/Patient |
| | | | Health Management App |
| | Vitamins/Minerals | Potassium/Sodium | Nutrition Database/Patient |
| | | | Health Management App |
| | Vitamins/Minerals | Calcium/Magnesium | Nutrition Database/Patient |
| | | | Health Management App |
| | Vitamins/Minerals | PRAL Alkalinity | Nutrition Database/Patient |
| | | | Health Management App |
| | Metabolic | Improve BP | Nutrition Database/Patient |
| | Improvers | | Health Management App |
| | Metabolic | Improve Cholesterol | Nutrition Database/Patient |
| | Improvers | | Health Management App |
| | Metabolic | Reduce Weight | Nutrition Database/Patient |
| | Improvers | | Health Management App |
| | Metabolic | Improve Renal | Nutrition Database/Patient |
| | Improvers | function | Health Management App |
| | Metabolic | Improve Liver | Nutrition Database/Patient |
| | Improvers | function | Health Management App |
| | Metabolic | Improve Thyroid | Nutrition Database/Patient |
| | Improvers | function | Health Management App |
| | Metabolic | Improve Arthritis | Nutrition Database/Patient |
| | Improvers | | Health Management App |
| | Metabolic | Reduce uric acid | Nutrition Database/Patient |
| | Improvers | | Health Management App |
| | Food Type | Fruits | Nutrition Database/Patient |
| | | | Health Management App |
| | Food Type | Oils | Nutrition Database/Patient |
| | | | Health Management App |
| | Food Type | Spices | Nutrition Database/Patient |
| | | | Health Management App |
| | Food Type | Grains | Nutrition Database/Patient |
| | | | Health Management App |
| | Food Type | Legumes | Nutrition Database/Patient |
| | | | Health Management App |
| | Food Type | Nuts | Nutrition Database/Patient |
| | | | Health Management App |
| | Food Type | Seed Products | Nutrition Database/Patient |
| | | | Health Management App |
| | Cellular | Inflammatory index | Nutrition Database/Patient |
| | Stressors | | Health Management App |
| | Cellular | Oxidative stress index | Nutrition Database/Patient |
| | Stressors | | Health Management App |
| | Cellular | Gluten | Nutrition Database/Patient |
| | Stressors | | Health Management App |
| | Cellular | Lactose | Nutrition Database/Patient |
| | Stressors | | Health Management App |
| | Cellular | Alcohol | Nutrition Database/Patient |
| | Stressors | | Health Management App |
| | Cellular | Allergic index | Nutrition Database/Patient |
| | Stressors | | Health Management App |
| | Hydration | Water | Nutrition Database/Patient |
| | | | Health Management App |

### IV. Patient Health Management Platform

IV.A General System Architecture

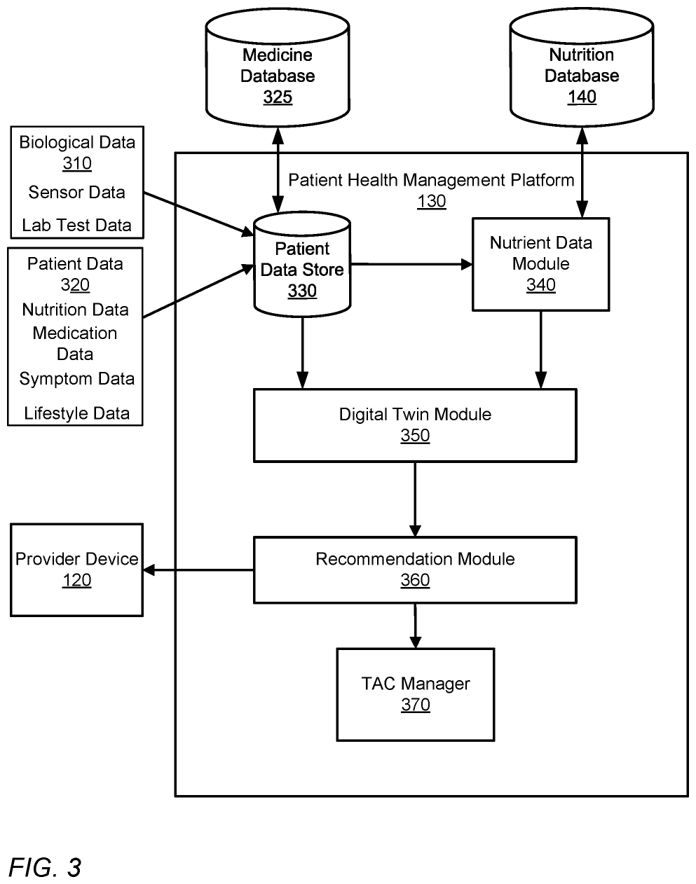

FIG. 3 is a block diagram of the system architecture of the patient health management platform 130, according to one embodiment. The patient health management platform 130 includes a patient data store 330, a nutrient data module 340, a digital twin module 350, a recommendation module 360, and a TAC manager 370. However, in other embodiments, the patient health management platform 130 may include different and/or additional components.

The patient health management platform 130 receives biological data 310 recorded by a variety of technical sources. Biological data 310 includes sensor data comprising biosignals recorded by one or more sensors worn or implemented by a patient. Such biosignals are continuously recorded and each recorded biosignal is assigned a timestamp indicating when it was recorded. Biological data 310 further includes lab test data determined based on blood draw analysis and/or other examinations that a patient has been subjected. Biosignals collected through lab test data may be recorded less frequently than biosignals collected through sensor data, for example over bi-weekly or monthly intervals. In some implementations, lab test data is determined based on procedures and analysis performed manually be doctors or researchers or based on analysis performed by machines and computers separate from the metabolic health manager 1000. The patient data store 330 stores biological data 310.

The patient health management platform 130 also receives patient data 320 that is recorded manually by a patient via an application interface on a patient device 110. Patient data 320 includes nutrition data, medication data, symptom data, and lifestyle data. Nutrition data describes a record of foods that a patient has consumed. In some implementations, nutrition data also includes a timestamp indicating when each food was consumed by the patient and a quantity in which each food was consumed. Similarly, medication data describes a record of medications that a patient has taken and, optionally, a timestamp indicating when a patient took each medication and a quantity in which each medication was taken. In response to a patient recording medication data, the patient health management platform may access additional information from a medication database (not shown) to supplement the medication data recorded by the patient. Symptom data describes a record of symptoms experienced by a patient and a timestamp indicating when each symptom was experienced. Lifestyle data describes a record of a patient's physical activity (e.g., exercise) and a record of a patient's sleep history. Lifestyle data may also include a description or selection of emotions or feelings capturing the patient's current state of mind and body (i.e., tired, sore, energetic). In one implementation, each type of patient data 320 may be recorded instantaneously throughout the day when the patient consumes a food, takes a medication, experiences a symptom, or experiences a change in an aspect of their lifestyle. In an alternate implementation, at the end of a day, the patient health management platform 130 detects that a patient has not instantaneously recorded patient data throughout the day and prompts the patient to input a complete record of patient data for the entire day at that time. In addition to biological data 310, the patient data store 330 stores patient data 320.

In some embodiments, the patient data store 330 stores biological data 310 and patient data 330 as an ongoing recorded timeline of entries for a current time period. As new patient data or biological data is recorded or as updates to existing patient data and biological data are received, the patient data store 330 updates the timeline of entries to reflect the new or updated data. Accordingly, the timeline of entries stored in the patient data store 330 comprises foods consumed by the patient at recorded times over the current time period, medications taken by the patient at recorded times over the current time period, and symptoms experienced by the patient at recorded times over the current time period. Some patient data entries may be recorded and reflected in the timeline on a daily basis, whereas other entries are recorded by a patient multiple times a day. Entries for biological data 310, for example, lab test data may be recorded even less frequently, for example as weekly updates to the ongoing timeline. The range of time between a start time and an end time for the current time period may be adjusted manually or trained over time based on predicted and true metabolic states for a patient.

The nutrient data module 340 receives nutrition data from the patient data store 330 and communicates the nutrition data to the nutrition database 140. As described above with reference to FIG. 1, the nutrition database 140 includes comprehensive nutrition information comprising macronutrient information (e.g., protein, fat, carbohydrates), micronutrient information (e.g., Vitamin A, Vitamin B, Vitamin C, sodium, magnesium), and biota nutrients (e.g. Lactococcus, Lactobacillus) for a wide variety of foods and ingredients. In some implementations, the nutrient data module 340 stores nutrition information in a lookup table or combination of lookup tables organized by food item or a category of food item. In other implementations, the nutrient data module 340 stores nutrition information in a lookup table organized by nutrient information or another suitable system. Based on the nutrition data received from the patient data store 330, the nutrient data module 340 identifies nutrition information associated with each food item of the nutrition data and supplements the nutrition data in the patient data store 330 with the identified nutrition information from the nutrition database 140. In some implementations, the nutrient database 140 includes over 100 food-related attributes including, but not limited to, different types of fat, protein, vitamins, and minerals.

The digital twin module 350 generates a digital replica of the patient's metabolic health based on a combination of biological data 310 and patient data 320, hereafter referred to as a digital twin. The digital twin module 350 considers different aspects of a patient's health and well-being to generate and continuously update a patient's digital twin. As described herein, a digital twin is a dynamic digital representation of the metabolic function of a patient's human body. The digital twin module 350 continuously monitors biological data and patient data and correlates a patient's metabolic history with their ongoing medical history to identify changes in the patient's metabolic state. In one embodiment, the digital twin module implements two sets of trained machine-learned metabolic models: a first set of models trained to predict the patient's metabolic state given patient data as inputs and a second set of models trained to determine the patient's true metabolic state given biological data as inputs.

Based on nutrition data, medication data, symptom data, lifestyle data, and supplemental nutrition information retrieved by the nutrient data module 340, the digital twin module 350 generates a prediction of the patient's metabolic state (herein referred to as a patient's “predicted metabolic state”). The digital twin module 350 implements one or more machine-learned, metabolic models to analyze the patient data 320 recorded over a given time period to generate a prediction of the patient's metabolic state for that time period. Accordingly, the prediction of the patient's metabolic state is a function of a large number of metabolic factors recorded in the patient data 320 (e.g., fasting blood glucose, sleep, and exercise) and a nutrition profile (e.g., macronutrients, micronutrients, biota nutrients).

In addition to the predicted metabolic state, the digital twin module 350 may implement one or more metabolic models to generate a true representation of a patient's metabolic state (herein referred to as a “true metabolic state”) based on the biological data 310 recorded for a time period. In comparison to the metabolic models used to generate a prediction of a patient's metabolic model, the metabolic models implemented by the digital twin module 4350 to determine the true metabolic state of the patient are trained to process aspects of biological data 310 (e.g., wearable sensor data and lab test data) into an affect the patient's metabolic state. For such implementations, at the conclusion of a time period, a metabolic model may be trained to analyze biological data 310 recorded by wearable sensors during the time period and determined based on lab tests from the time period to determine a true metabolic state for the patient that reflects the actual biological conditions experienced by a patient (e.g., their HbA1c levels, or BMI) during the time period. Accordingly, given biological data 310 as an input, the metabolic model is further trained to output a patient's actual biological response (e.g., a measured insulin sensitivity or change in glucose in response to consuming a food or taking a medication).

In some embodiments, digital twin module 350 communicates both the predicted metabolic state and the true metabolic state to the timeliness, accuracy, and completeness (TAC) manager 370. The TAC manager 370 compares the predicted metabolic state and the true metabolic state to determine whether the two states are within a threshold level of similarity to each other. If the two states are within a threshold level of similarity, the TAC manager 370 confirms the timeliness, accuracy, and completeness of the recorded patient data. As described herein, accurately recorded nutrition data, medication data, symptom data, and lifestyle data is accurate in what was recorded in the entry and when the entry was recorded.

Alternatively, if the two states are not within a threshold level of similarity, the TAC manager 370 detects that there is an error in the record of the patient data 320. Examples of such errors detected by the TAC manager 370 include, but are not limited to, an entry recorded in an incorrect amount, a failure to record an entry, or an entry recorded at the wrong time. Based on the inconsistency, or inconsistencies, between the true metabolic state and the predicted metabolic state, the TAC manager 370 identifies one or more potential errors in the recorded patient data which may have contributed to the one or more inconsistencies and generates notifications to the patient device 110 for presentation to the patient.

Patient data and biological data may be recorded at varying intervals. For example, sensor data is recorded continuously every 15 minutes, lab test data is recorded bi-weekly, and patient data 320 is recorded multiple times a day as needed. Therefore, the patient health management platform 130 may not receive an updated recording for every type of data in time to generate a predicted metabolic state. When generating a predicted metabolic state for a particular time period, the digital twin module 350 retrieves all patient data 320 recorded within that time period and the metabolic state predicted by the during the preceding time period. In some embodiments, the digital twin module 350 implements one or more machine learning models to process, as inputs, the recorded patient data and the most recently predicted metabolic state into a predicted metabolic state for a current time period. In place of the most recent predicted metabolic state, the digital twin module 350 may input the most recent true metabolic state to the one or more machine learning models. Accordingly, the predicted metabolic state reflects any effects that the most recently recorded patient data 320 had on a previous metabolic state.

Similarly, when generating a true metabolic state for a time period, the digital twin module 350 retrieves all biological data 310 recorded within that time period (e.g., heart rate, exercise, continuous blood glucose, ketones, blood pressure, weight) and the true metabolic state generated during the preceding time period. The digital twin module 350 may also rely on one or more machine learning models to process the retrieved biological data 310 and the most recent true metabolic state into a current true metabolic state. Accordingly, the generated true metabolic state also reflects any effects of the most recently recorded biological data 310 had on a previous metabolic state. For example, a machine learned model may use a continuous blood glucose signal measured every 15 minutes to calculate a patient's 5-day average blood glucose. The computed measurement is compared against established ranges in the medical literature to determine whether the patients are in a diabetic, pre-diabetic, or non-diabetic state as they progress with their treatment. In common implementations, the digital twin module 350 updates a patient's metabolic state at a higher frequency than a frequency at which lab test data is recorded. As such, when lab test data is unavailable for the current time period, the digital twin module 350 may generate the updated metabolic state based on the lab test data recorded most recently for a preceding time period.

In one embodiment, the recommendation module 360 compares a patient's predicted metabolic state to baseline metabolic state for a patient with a functional metabolism. For patients who already have a functional metabolism, the recommendation module 360 compares the predicted metabolic state to a baseline metabolic state for a patient with an optimal metabolism. In either implementation, the recommendation module 360 determines discrepancies between the patient-specific predicted metabolic state and the baseline metabolic state and identifies one or more biosignals which could be adjusted such that the predicted state becomes more similar to the baseline state, for example lower blood glucose levels in the predicted metabolic state or an imbalance between certain micronutrients and micronutrients.

Based on the determined adjustments, the recommendation module 360 generates a recommendation for improving the patient's biosignals to more closely resemble those of the baseline metabolic state. The recommendation includes a set of objectives for a patient to complete to improve the patient's metabolic health. The set of objectives include a medication regimen or schedule, a food or meal schedule, micronutrient and biota nutrient supplements, one or more lifestyle adjustments, or a combination thereof. The medication regimen, food schedule, and supplement schedule may prescribe medications, food items, or supplements which may either replenish nutrients in which a patient is deficient, offset the effects of nutrients for which a patient has an excess, or a combination thereof. The medication regimen, food schedule, and supplement schedule may also alleviate or mitigate the symptoms (as indicated by symptom data recorded by a patient) that a patient is experiencing by addressing the biological root cause of the symptoms. One example of a medication regimen may include a recommended medication or combination of medications and an adherence schedule for each medication. One example of a food schedule may include a recommended food item or, more broadly, a category of food item and an amount of the food item to be consumed. Similarly, a lifestyle adjustment may prescribe particular lifestyle adjustments for addressing a patient's symptoms or nutrient abnormalities. Examples of lifestyle adjustments include, but are not limited to, increasing physical activity or increasing a patient's amount of sleep. In some implementations, the content of lifestyle adjustments may broadly overlap with food or medication adjustments. For example, a lifestyle adjustment may recommend a patient replace refined carbohydrates with wholegrain foods, while the food schedule includes a set of particular wholegrain foods.

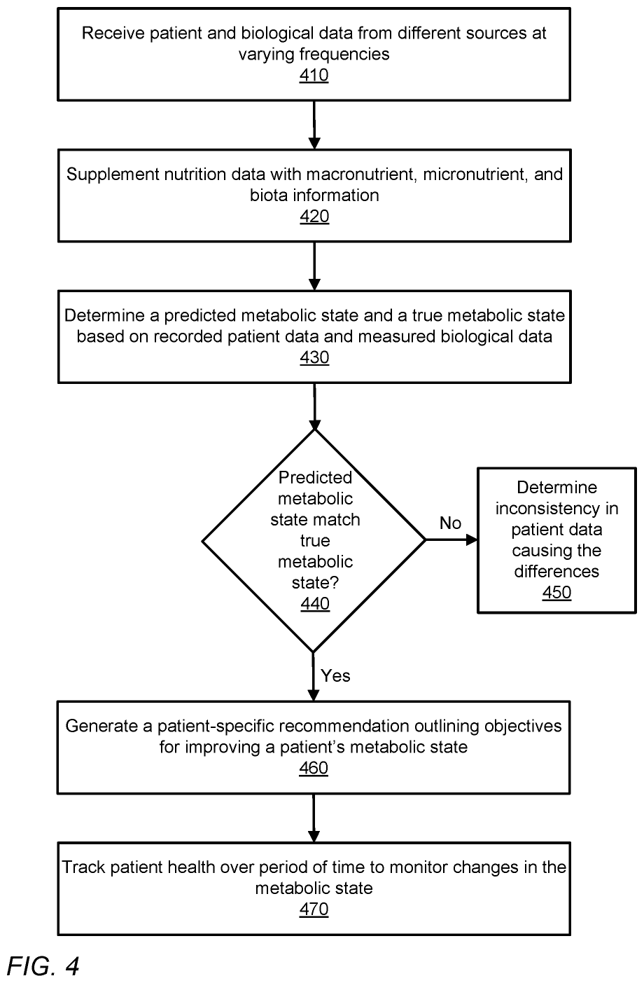

FIG. 4 is a flowchart illustrating a process for generating a patient-specific recommendation for improving metabolic health of a patient, according to one embodiment. The patient health management platform 130 receives 410 patient data and biological data from different sources at varying frequencies. Patient data describes data manually recorded by a patient and communicated to the platform 130. Biological data describes data manually recorded by wearable sensors or measured based on lab tests before being communicated to the platform 130.

Patient data includes nutrient data which is recorded by the patients as a list of foods which have been consumed by the patient over a time period. While the impact of a food item by itself on a patient's metabolic state may not be known, the impact of particular macronutrients, micronutrients, and biota nutrients associated with the food item on a patient's metabolic state is known. As a result, the patient health management platform 130 accesses a nutrition database 140 storing such macronutrient, micronutrient, and biota nutrient information. Based on the accessed information, the platform 130 supplements 420 the recorded nutrition data with the accessed macronutrient, micronutrient, and biota information.

Consistent with the description above in Section IV.A, the platform 130 determines 430 a predicted metabolic state based on the recorded patient data (e.g., patient data 320) and the patient's true metabolic state based on the measured biological data (e.g., biological data 310). The platform 130 compares 440 the predicted metabolic state with the true metabolic state to determine whether the two states match, or are within a threshold level of similarity. If the two metabolic states are not within the threshold level of similarity, the platform 130 determines 450 one or more inconsistencies in the patient's recording of their patient data, which may have caused the predicted metabolic state to differ from the true metabolic state. The platform 130 communicates the inconsistency back to a patient device (i.e., patient device 110). Upon receiving the inconsistency, the patient device 110 presents a user interface notifying the patient of the inconsistency and enabling the patient to correct the inconsistency. The platform 130 receives the updated patient data and determines 430 an updated predicted metabolic state based on the updated patient data.

If the two metabolic states are within a threshold level of similarity, the platform 130 categorizes the predicted metabolic state as representative of poor metabolic health, functional metabolic health, or optimal metabolic health. Based on the assigned category, the platform 130 generates 460 a patient-specific recommendation outlining objectives for improving the patient's metabolic state. In particular, the recommendation may outline objectives for consuming food, taking medication, or engaging in lifestyle adjustments to supplement nutrients in which a patient is deficient and that may have contributed to the patient's deteriorated metabolic state.

Following the receipt of the recommendation, a patient continues to record patient data and wearable sensors continue to record biological data, both of which are representative of a metabolic state for a subsequent time period. As patient data and biological data continue to be recorded, the patient health management platform 130 tracks 470 patient health over a time period to monitor changes in the patient's metabolic state. Based on the monitored changes, the platform 130 is able to confirm whether or not a patient is adhering to the recommendation generated by the platform 130. If the patient is not adhering to the recommendation, the platform 130 may generate a notification or reminder to the patient, a doctor assigned to the patient, a coach assigned to the patient, or a combination thereof. If that patient adheres to the recommendation, the platform 130 is able to review the changes in metabolism to confirm that the recommendation is improving the patient's metabolic health. If the platform is not improving the patient's metabolic health, the platform 130 is able to dynamically revise the recommendation to correct the deficiencies of the initial recommendation. If the platform is improving the patient's metabolic health, the platform 130 is able to dynamically update the recommendation to continue to optimize the patient's metabolic health in view of their improved metabolic state.

More information regarding the patient health management platform 130 and its components, as well as the interactions between those components, can be found in U.S. patent application Ser. No. 16/993,177, filed Aug. 13, 2020, U.S. patent application Ser. No. 16/992,184, filed Aug. 13, 2020, and U.S. patent application Ser. No. 16/993,189, filed Aug. 13, 2020, each of which are incorporated by reference herein in their entirety.

IV.C Metabolic Digital Twin

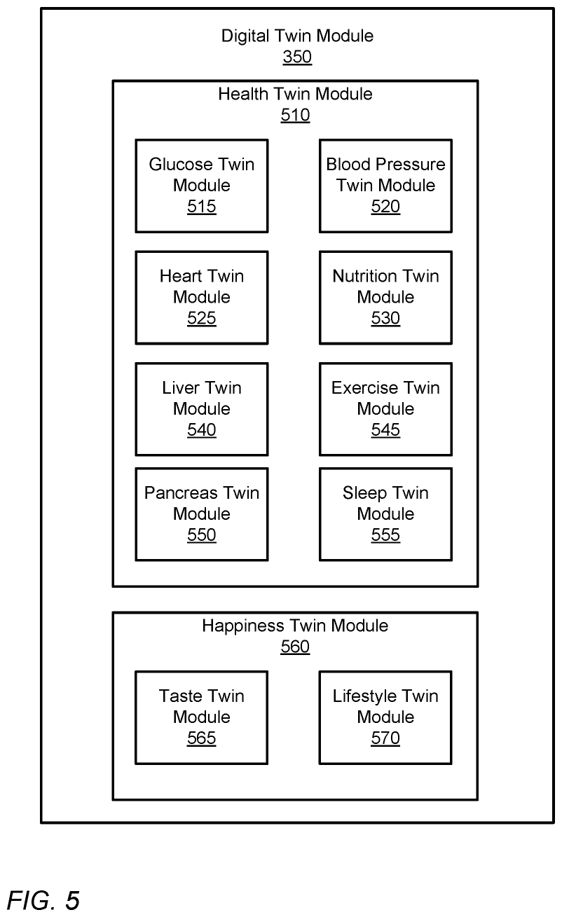

As described above, the digital twin module 350 generates a digital twin of the patient's metabolic health to continuously monitor and update different aspects of a patient's health and well-being. FIG. 5 is a block diagram of the system architecture of a digital twin module 350, according to one embodiment. The digital twin module 350 includes a health twin module 510 and a happiness twin module 560. The digital twin module 350 may include different and/or additional components to perform the same functions described with regards to the digital twin module 350. The digital twin module 350 generates a digital replica of a patient's metabolic state in two dimensions: a health dimension and a happiness dimension.

The health twin module 510 generates a digital replica of the health dimension of a metabolic state based on biological measurements recorded by wearable sensors and lab test data. In the embodiment illustrated in FIG. 5, the health twin module 510 comprises a glucose twin module 515, a blood pressure twin module 520, a heart twin module 525, a nutrition twin module 530, a liver twin module 540, an exercise twin module 545, a pancreas twin module 550, and a sleep twin module 555. Each component of the health twin module 510 captures and updates a critical aspect of a patient's metabolic health such that the digital twin represents the patient's overall metabolic health. The health twin module 510 may include additional, fewer, or a different combination of components to generate a digital twin based on varying aspects of a patient's metabolic health. In some embodiments, each component of the health twin module 510 generates an output indicating a condition of an aspect of the patient's metabolic health. For example, the heart twin module 525 may generate an output indicating the patient's heart health rating on a scale of 100, for example 85. This is derived from cardiac health biomarkers such as Lipoprotein(a), Apolipoprotein B, and High-Sensitivity C-Reactive Protein (HS-CRP).

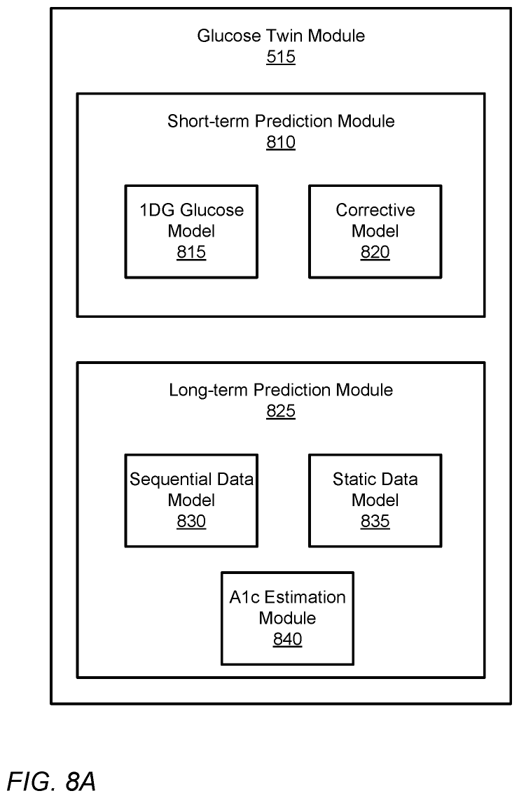

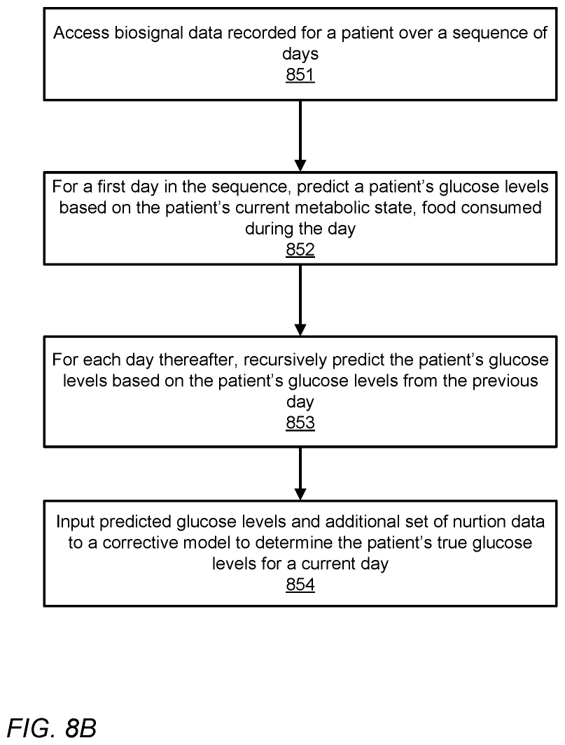

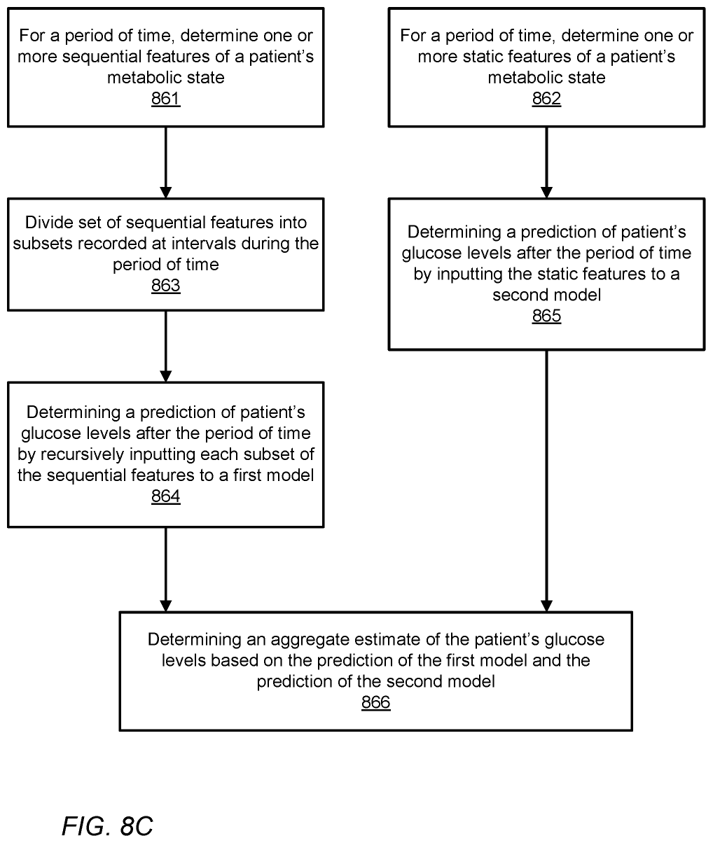

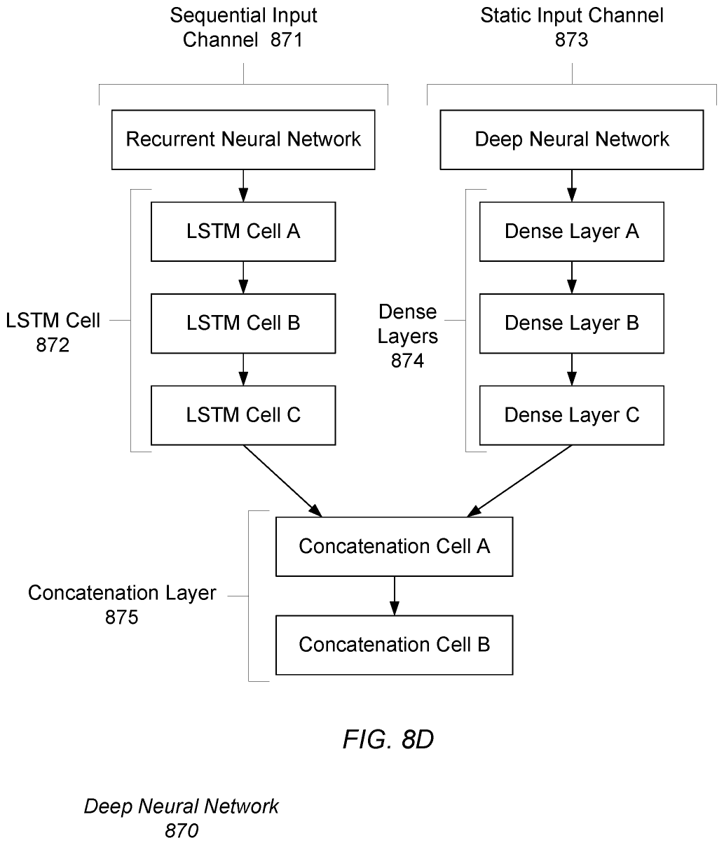

The glucose twin module 515 tracks and analyzes glucose dynamics for a patient over time to enable the digital twin to model glucose dynamics for the patient. The glucose twin module 515 may analyze glucose dynamics recorded via a wearable sensor. The heart twin module 525, liver twin module 540, and the pancreas twin module 550 track and analyze function and physiology of a patient's heart, liver, and pancreas to enable the digital twin to model heart, liver, and pancreas function for the patient. The heart twin module 525, the liver twin module 540, and the pancreas twin module 550 may analyze function of a patient's heart, liver, and pancreas based on information recorded via one or more lab tests. The blood pressure twin module 520 tracks and analyzes blood pressure dynamics for a patient over time to enable the digital twin to model blood pressure dynamics for the patient. The blood pressure twin module 515 may analyze blood pressure dynamics recorded via a wearable sensor or via lab test data. The glucose twin module 515 is further described with reference to FIGS. 8A-D.

The nutrition twin module 530 communicates with the nutrient data module 530 to track and analyze nutrition information of food consumed by a patient to enable the digital twin to model the impact of food consumed by the patient. The nutrition twin module 530 may analyze a combination of macronutrient parameters, micronutrient parameters, and biota nutrients for each food item recorded by the patient through a patient device 110. The exercise twin module 545 tracks exercise activity for a patient and analyzes those exercise habits by correlating periods of exercise (or inactivity) with changes in the patient's metabolic state. The exercise twin module 545 may analyze exercise activity recorded by the patient through a patient device 110. Similarly, the sleep twin module 555 tracks sleep trends for a patient and analyzes those sleep trends by correlating quality, length, and frequency of sleep with changes in the patient's metabolic state. The sleep twin module 555 may analyze sleep trends recorded by the patient through a patient device 110 or by a wearable sensor.

Each module (or component) of the health twin module 510 is connected to and communicates with other modules of the health twin module 510 to capture the complex interaction effects that contribute to a patient's metabolic state. For example, blood pressure dynamics are driven by a combination of factors including blood glucose dynamics, heart function, nutrition, exercise, and sleep trends. Each of those driving factors are, in turn, driven by other factors represented in the patient's digital twin.

The happiness twin module 560 generates a digital replica of the happiness dimension of a patient's metabolic state based on feedback recorded through a patient device 110. In the embodiment illustrated in FIG. 5, the happiness twin module 560 comprises a taste twin module 565 and a lifestyle twin module 570. Each component of the happiness twin module 560 captures a critical aspect of a patient's satisfaction with their recommended treatment to their digital twin such that the digital twin also represents the patient's overall experience with treatment. The happiness twin module 560 may include additional, fewer, or a different combination of components to generate a digital twin based on varying aspects of a patient's metabolic health. In some embodiments, each of the taste twin module 565 and the lifestyle twin module 570 generate an output indicating a patient's current state of mind regarding a food item, meal recommendation, or a lifestyle recommendation prescribed by a patient-specific recommendation. For example, each food consumed by a patient may be labeled with a score on a 5-star scale, such as “4 stars”.

The taste twin module 565 communicates with the nutrition twin module 530 to assign a preference to each food item recorded by the patient (e.g., a label indicating whether the patient enjoyed the food item or not). In conjunction, the nutrition twin module 530 and the taste twin module 565 may compare two foods with a similar metabolic effect and prioritize whichever food the patient enjoyed more. The food item that the patient enjoyed more will be carried forward in other future patient-specific recommendations. The lifestyle twin module 570 communicates with the exercise twin module 545 and the sleep twin module 555 to assign a preference to the activities recorded by the patient. For example, if a patient wishes to engage in more exercise, future treatment recommendations may be generated with an emphasis on more frequent exercise.

As described herein, each module of the health twin module 510 and the happiness twin module 560 includes a uniquely trained metabolic model. In particular, when generating a prediction of a patient's metabolic state, each involved metabolic model is trained to determine an impact of a particular type of patient data input on a patient's metabolic state. When generating a prediction of a patient's metabolic state, the digital twin module 350 may consider the output of the metabolic models trained to receive patient data as inputs, for example the nutrition twin module 530, the exercise twin module 545, the sleep twin module 555, and the lifestyle twin module 570. For example, the nutrition twin module 530 implements a metabolic model to predict a patient's metabolic state based on patient data identifying food items consumed by the patient. As additional examples, each of exercise twin module 545, the sleep twin module 555, and the lifestyle twin module 570 implement a metabolic model to predict a patient's metabolic state based on patient data describing the patient's exercise habits, sleep habits, and lifestyle habits, respectively. The digital twin module 350 may also consider metabolic models that are not illustrated in FIG. 5, or the other twin modules that are illustrated in FIG. 5 when generating a prediction of a patient's metabolic state.

In comparison, each metabolic model involved in determining a patient's true metabolic state is trained to determine an impact of a particular type of biosignal input on a patient's metabolic state, for example the glucose twin module 515, the blood pressure twin module 520, the heart twin module 525, the liver twin module 540, and the pancreas twin module 550. For example, the glucose twin module 515 implements a metabolic model to evaluate a patient's true metabolic state based on input biosignals describing the glucose dynamics of the patient. As additional examples, each of the heart twin module 525, the liver twin module 540, and the pancreas twin module 550 implement metabolic models to evaluate, respectively, a true performance of a patient's heart, liver, and pancreas based on input biosignals describing the functionality of those organs. As yet another example, the blood pressure twin module 520 implements a metabolic model to evaluate a patient's true metabolic state based on input biosignals describing blood pressure dynamics of the patient. The digital twin module 350 may also consider metabolic models that are not illustrated in FIG. 5, or the other twin modules that are illustrated in FIG. 5 when generating a prediction of a patient's metabolic state.

In some embodiments, modules of the digital twin module 350 may implement a combination of multiple machine-learned models to more accurately and completely characterize each aspect of a patient's metabolic health. For example, as will be described below in Section IV.D, the glucose twin module 515 may implement both a glucose impact model (as described in Section IV.D.1) and a 1-Day Average Glucose model (as described in Section IV.D.2).

After a digital twin of a patient has been initialized, components of the digital twin module 350 continuously collect data describing changes in conditions contributing to the patient's metabolic health. When any component of the digital twin module 350 receives updated data, the digital twin module 350 updates a digital twin of the patient in near real-time to reflect the updated data.

IV.D Machine-Learned Metabolic Models

Because the human body is a complex system and different patients may respond differently to the same input stimuli, the patient health management platform 130 includes mathematical models trained to learn the relationships between response signals representing a patient's metabolic states and input stimuli causing those responses. As described above, the patient health management platform 130 applies machine-learning based artificial intelligence to generate a precision treatment recommendation for improving a patient's metabolic health by predicting their response to future input stimuli. The digital twin module 350 implements a combination of machine-learned models that are iteratively trained to predict a response of the human body based on each patient's current metabolic state and a set of inputs (e.g., recorded patient data, sensor data, and biological data). Each machine-learned model enables the digital twin module 350 to automatically analyze a large combination of biosignals recorded for each patient to characterize a patient's current or potential metabolic state.

In order to model a patient's metabolic state and to track changes in their metabolic health, a model, such as a mathematical function or other more complex logical structure, is trained using the combination of input biosignals described above, to determine a set of parameter values that are stored in advance and used as part of the metabolic analysis. Briefly, a representation of a patient's metabolic state is generated by inputting wearable sensor data, lab test, and recorded patient data as input values to the model's function and parameters, and, together with values assigned to those parameters, determines a patient's metabolic health. As described herein, the term “model” refers to the result of the machine learning training process. Specifically, the model describes the generation of a function for representing a patient's metabolic state and the determined parameter values that the function incorporates. “Parameter values” describe the weight that is associated with at least one of the featured input values. “Input values” describe the variables of the function or the conditions to be used in conjunction with the parameter values to determine the risk score. Input values can be thought of as the numerical representations of the various features that the model takes into account, for example the input biosignals. During training, from input values of the training dataset, the parameter values of a model are derived. Further, the training data set is used to define the parameter values at a specified time interval, whereas the input values are continuously updated by the patient's conditions.

The digital twin module 350 may include a combination of machine-learned models to generate various representations of a metabolic state, for example metabolic models trained to predictively model a patient's metabolic state based on recorded nutrition data, medication data, symptom data and lifestyle data, and to model a patient's true metabolic state based on sensor data and lab test data. The digital twin module 350 may input patient data 320, for example nutrition data, medication data, symptom data, or lifestyle data, into a combination metabolic models (e.g., the nutrition twin model 530 and the lifestyle twin module 570) to predict a patient's metabolic state that would result from the recorded patient data. The digital twin module 350 may compare a recorded timeline of patient data (e.g., foods consumed by the patient, medications taken by the patient, and symptoms experienced by the patient) during a time period to a metabolic state generated for the time period to determine an effect of each food item, medication, and symptom on the metabolic state of the patient.

Additionally, the digital twin module 350 may implement one or more metabolic models to predict a patient's metabolic state that would result from the recommended nutrition, medication, or lifestyle changes included in a recommendation. Alternatively, the digital twin module 350 may receive biological data, for example sensor data and lab test data, as inputs to metabolic models to determine a patient's actual metabolic response to the patient data 320.

Each metabolic model is trained using a training dataset made up of large volumes of historical patient data and biological data recorded for a significant volume of patients, respectively. The training set includes daily metabolic inputs and corresponding daily metabolic outputs. Inputs, for example, include patient data 320 recorded for a current time period (i.e., different foods, medication, sleep, exercise, etc.) and a patient's initial metabolic state before the patient data 320 was recorded (e.g., based on biosignals derived from sensor data and lab test data). Inputs measured by wearable sensors and lab tests or recorded manually by a patient may be encoded into a vector representation, for example a feature vector, that a machine-learned model is configured to receive. A feature vector comprises an array of feature values each of which represents a measured or recorded value of an input biosignal.

Outputs, for example, include the actual biological data 310, which represents biosignals characterizing a patient's metabolic health (i.e., blood glucose level, blood pressure, and cholesterol). These act as baseline models trained on historical data that can then be applied to new patients with metabolic issues needing treatment to make predictions about those new patients based on what the models have learned from historical patients. Once trained, the machine-learned model may be applied to predict new metabolic states for the new patients based on new combinations of biosignals to predict how a novel set of input biosignals would result in different output signals, for example lowering blood glucose to improve diabetes or lowering blood pressure to improve hypertension.

The models are iteratively trained by feeding the input biosignals and metabolic state outcomes for existing and new patients into these models such that the models continue to learn and are continuously updated based on these new data points. For example, after a metabolic state model determines an aspect of a patient's true metabolic state for a time period, the digital twin module 350 may update a training dataset with the determined true metabolic state and a plurality of biosignals recorded during the time period that contributed to the true metabolic state. The metabolic state model(s) are periodically re-trained based on the updated training dataset. This continuously improves the model and allows it to accurately predict future metabolic states for each patient based on their biosignal inputs. In comparison, the metabolic state model is trained or re-trained/modified on a training dataset comprising the information described above for a particular patient.

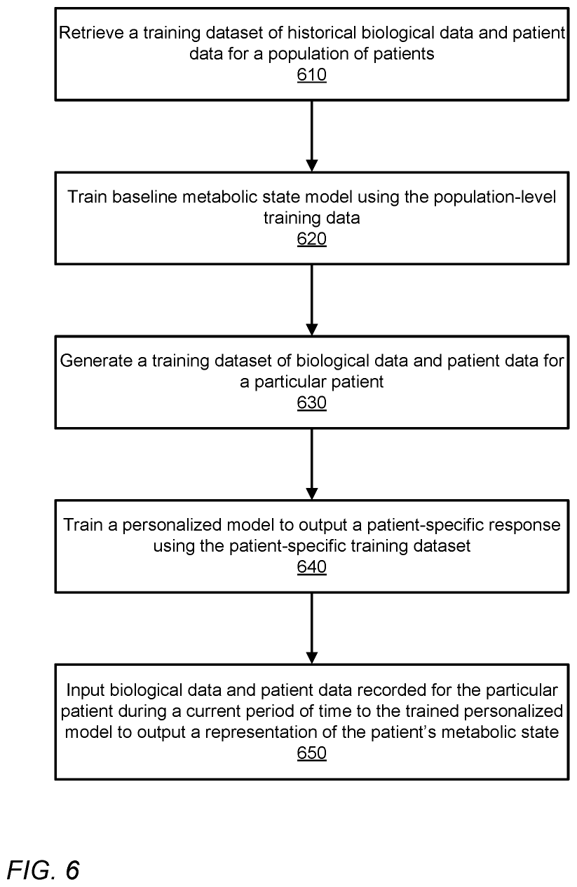

FIG. 6 is an illustration of the process for training a machine-learned model to output an aspect of a patient's metabolic health, according to one embodiment. The digital twin module 350 retrieves 610 a training dataset comprised of historical biosignals (e.g., historical sensor data and lab test data) and patient measured and/or recorded for an entire population of patients. Each historical measurement of biological data and record of patient data is assigned a timestamp representing when the patient experienced the measurement/recording and a label identifying its impact on a patient's metabolic health, the patient's metabolic response to the measurement, or both. Using the training dataset of population-level data, the digital twin module 350 trains 620 a baseline model. The training dataset of population-level data comprises labeled metabolic states recorded for a population of patients and sensor data and lab test data that contributed to each labeled metabolic state. Once trained, the baseline model may be implemented to determine a metabolic state of a representative patient of the population of patients (e.g., an average patient) given a set of biological inputs, for example biological data or patient data.

In some implementations, the baseline model may be further trained to generate a personalized representation of a patient's metabolic health. In such implementations, the digital twin module 350 generates 630 an additional training dataset of biological data and patient data for a particular patient. The digital twin module 350 accesses both measured biological data and recorded patient data for a particular patient and aggregates that data into a training dataset. Similar to the historical training dataset, the biological data and patient data of the training dataset are assigned a timestamp and a label to characterize how each biological input impact the particular patient's metabolic state. Using the training dataset of patient-specific data, the digital twin module 350 trains 640 a personalized metabolic model. Once training, biological data and patient data recorded during a subsequent time period may be input 650 to the trained model to output a representation of a particular patient's metabolic state.

Depending on the type of data input to either the personalized or baseline metabolic model, the digital twin module 350 may generate a representation of a patient's true metabolic state or their predicted metabolic state. Biological data, for example data recorded by a wearable sensor or a lab test, may be input to a model to generate a representation of a patient's true metabolic state consistent with the description above. Alternatively, patient data, for example nutrition data, medication data, symptom data, and lifestyle data, may be input to a model to generate a prediction of patient's current metabolic state consistent with the description above.

Training both models in such a manner enables the patient health management platform 130 to predict a patient's metabolic response to future input stimuli (i.e., patient data 320 recorded by a patient in the future) for not just patients already included in the training dataset, but also new patients included in a holdout dataset because the model only relies on the knowledge representing a patient's current metabolic state and the patient's input stimuli to predict their patient-specific response. Additionally, the model predicts a patient's response to input stimuli for each patient at different stages of his or her treatment because the platform maintains a history of a patient's changing metabolic condition. Finally, it allows for long-range precision prediction of the patient's metabolic state by using current and short-range predictions to inform longer-range predictions.

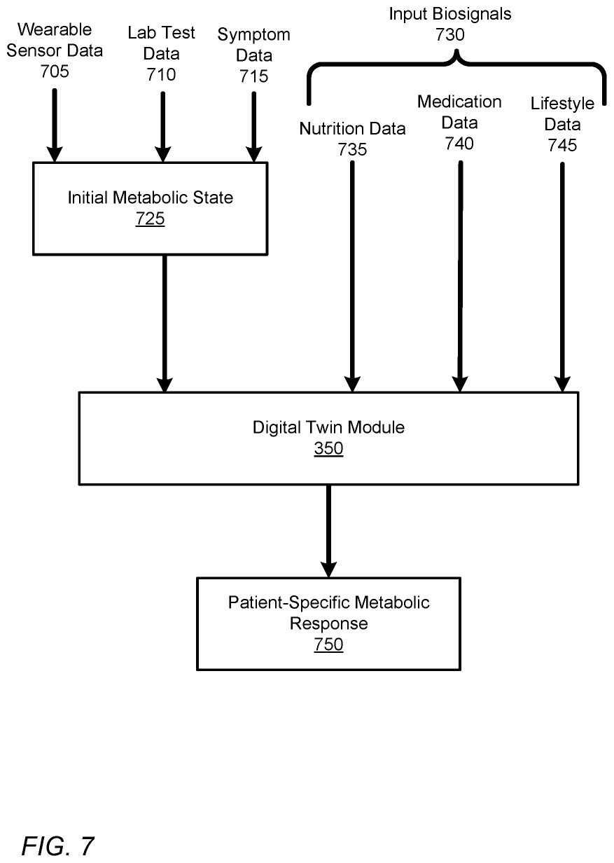

FIG. 7 is an illustration of the process for implementing a machine-learned model, according to one embodiment. For a given time period, biosignals recorded as wearable sensor data 705, lab test data 710, and symptom data 715 are representative of a patient's actual, current metabolic state. Accordingly, based on these input biosignals, the patient response module generates an initial metabolic state 725. When sufficient training data exists for a particular patient, the initial metabolic state 725 may be determined using a metabolic model(s). Alternatively, the initial metabolic state 725 may be determined using metabolic model(s) trained for a population of patients. Additionally, the digital twin module 350 relies on input biosignals 730, which represent biosignals that may impact a patient's metabolic state, either deteriorating or improving the state. For example, input biosignals 730 may include nutrition data 735, medication data 740, and lifestyle data 745 recorded for a patient at a time occurring after the generation of the initial metabolic state. In addition to the initial metabolic state 725, the digital twin module 350 receives the input biosignals 730 recorded by the patient as inputs one or more metabolic models. Accordingly, digital twin module 350 models the patient's patient-specific metabolic response 750 to the inputted biosignals. Described differently, the patient-specific metabolic response 750 represents one or more changes in a patient's initial metabolic state caused by, or at least correlated with, the input biosignals 730.

For a second time period following the determination of the patient-specific metabolic response 750, the platform 130 continues to record wearable sensor data 705, lab test data 710, and symptom 715. Given biosignals recorded as wearable sensor data 705 and lab test data 710 as inputs, the aggregated output of the combination of metabolic models (e.g., the true metabolic state) describes what a patient's metabolic response actually is during a time period. Given nutrition data 735, medication data 740, and lifestyle data 745 (e.g., input biosignals 730) recorded during the same time period as inputs, the aggregated output of the combination of metabolic models (e.g., the predicted metabolic state) describes what a patient's metabolic response should be during the time period. Accordingly, a comparison of the two outputs allows the platform 130 to verify the timeliness, accuracy, and completeness with which a patient recorded the input biosignals 730.

IV.D.1 Virtual Continuous Glucose Monitor

The glucose twin module 515 implements a combination of machine-learned models to generate predictions of a patient's glucose levels for various situations and conditions. FIG. 8A is a block diagram of the system architecture of a glucose twin module 515, according to one embodiment. The glucose twin module 515 includes a short-term prediction module 810 and a long-term prediction module 825. The glucose twin module 515 may include different and/or additional components to perform the same functions described with regards to the glucose twin module 515. The short-term prediction module 810 implements a combination of one or more machine-learned models to determine a regular (e.g., daily) prediction of a patient's glucose levels. The long-term prediction module 825 implements a second combination of one or more machine-learned models to determine a less frequent prediction, for example a weekly, monthly, or quarterly prediction, of a patient's glucose levels.

As described above, with reference to FIG. 6, both the short-term prediction module 810 and the long-term prediction module 825 apply a training dataset of historical blood glucose data from a population of patients to each machine-learned model to generate a baseline model. As described herein, each baseline model is trained to predict measures of blood glucose at the population level based on data, for example sensor data and lab test data, collected from the population. When a patient initializes a profile on the patient health management platform, the baseline models of one or both of the short-term prediction module 810 and the long-term prediction module 825 are further fine-tuned to the metabolic state of the patient to characterize the specific metabolic state and condition of the patient based on a personalized training dataset for the patient including sensor data and lab test data collected from the patient. Over the course of an initialization period (e.g., a patient's first 35 days using the platform 130), lab test data, sensor data, and patient data are collected from the patient and correlated with the patient's true glucose levels. During the initialization period, a patient may wear a physical CGM to monitor their true glucose levels, but after the initialization period the patient may stop wearing the physical CGM. Following the conclusion of the initialization period, sensor data may be recorded by non-invasive sensors worn by the patient, for example a fitness tracker or a fitness watch, which are configured to monitor the patient's heart rate and to track the patient's physical activity.

Using a combination of trained, patient-specific models, the glucose twin module 515 generates accurate estimations of a patient's blood glucose levels based on a combination of sensor data and lab test data recorded for the patient and/or nutrition data recorded by the patient. The glucose twin module 515 updates metabolic parameters of a digital twin of the patient's metabolic state (for example, as described above with reference to FIG. 5) in view of the most recent estimations of the patient's glucose levels.

The trained combination of one or more machine-learned models implemented by the glucose twin module 515, which may also be referred to herein as a “virtual CGM”, provides a superior patient experience compared to conventional CGM technologies. The virtual CGM described herein is non-invasive, requires no finger pricks, no implanted sensors, and minimal participation or input from the patient. The virtual CGM relies only on sensor data collected by one or more non-invasive sensors (e.g., a fitness watch) and optional food reporting, resulting in improved patient adherence compared to conventional CGMs. Additionally, software processing for the virtual CGM is inexpensive and the virtual CGM has no recurring hardware costs, which yields a significant cost improved, for example a 30× to 60× improvement, compared to conventional CGMs.

During the early stages of a patient's participation with the platform 130, the glucose twin module may implement machine-learned model(s) of the short-term prediction module 810 to closely track the immediate effects of the patient's adherence to a treatment recommendation on their metabolic state. Such close tracking provides additional insight, which the for platform 130 uses to generate a treatment recommendation for improving or maintaining the specific patient's metabolic state. Additionally, the short-term prediction module 810 may mandate that a patient report food consumed during the early stages of the patient's participating with the platform 130 to enable to the short-term prediction module 810 to generate daily glucose level predictions based, in part on nutrition data collected for the food consumed by the patient. In one implementation, such early stages refer to at least the first three months of the patient's participation on the platform 130. In alternate embodiments, the length of time of the early stages may be extended or shortened based on the improvement or deterioration of a patient's metabolic state.

In one embodiment, the short-term prediction module 810 implements a 1DG model 815 and a corrective model 820 to generate an estimate of a patient's average daily blood glucose. The 1DG model 815 predicts a patient's 1-Day Average Glucose (1DG) given the patient's metabolic state at the start of a time interval and nutrition data collected based on a record of food items consumed by the patient during the time interval. As described herein, the patient's 1DG describes their average blood glucose level over a 24-hour calendar day. The 1DG model 815 characterizes the metabolic state of the patient by predicting the average blood glucose level of the patient during a 24-hour calendar day based on a metabolic profile of the patient determined on the preceding day and all foods consumed by the patient during the current day. The metabolic profile of the patient may be determined based on a combination of factors including a patient's most recently recorded lab test data, sensor data, and foods consumed a preceding time period. For example, if a patient adheres to a seven-day long nutrition recommendation outlining particular food items to be eaten as breakfast, lunch, dinner, and snacks during those seven days, the 1DG model 815 predicts the patient's 1DG progression over those seven days based on the report of consumed food items and the patient's metabolic profile over the course of the seven days.

As discussed above, the digital twin module 350 determines a patient's initial metabolic state based on biological data including, but not limited to, HBa1c, fasting glucose, minutes asleep/awake, sleep efficiency, sedentary minutes, calories BMR, BMI, calories output, exercise calories output, metformin dosage, glimepiride dosage. The digital twin module 350 additionally considers nutrition data including, but not limited to, protein, fat, carbohydrates, fiber, net carbohydrates, net glycemic index carbohydrates, calories, and glycemic load for each recorded food item, as well as derived features created as ratios between nutrients and derived features representing ratios of nutritional to personal information may be used as well. The short-term prediction model may implement a highly parallelized optimization algorithm to identify the optimal combination of features for performance of the 1DG model 815. The long-term prediction module 825 may similarly implement a highly parallelized optimization algorithm to identify the optimal combination of features for performance the implemented machine learning models.

In one embodiment, the 1DG model 815 implements gradient boosting techniques (e.g., a GradientBoostingRegression from the Sci-Kit Library or the XGBoostRegressor from the XGBoostLibrary) in combination with a patient cohorting algorithm to predict a patient's 1DG and their resulting fasting glucose for a first day of a time period given a combination of metabolic features (e.g., the biological data discussed above) and nutrition data collected from food reported by the patient. The cohorting algorithm selects discrete subpopulations from an entire population of patients based on the metabolic similarity between each patient in the subpopulations and the patient whose metabolic state is being modeled. The 1DG model trains separate gradient boosting models based on each of these subpopulations. The 1DG model implements gradient boosting techniques to create n number of weak learners, or “trees,” where each tree is created to reduce the error from the combination of learners that preceded it.

In one embodiment, the 1DG model 815 is trained on 80% of the patient data randomly chosen after cleaning and filtering the entire history of patient data and is validated on the remaining 20% of the cleaned and filtered patient data. In such embodiments, the 1DG model 815 is trained to generate predictions with a Median Absolute Error of less than 3.5.

The 1DG model 815 predicts a patient's 1DG and fasting glucose for a current day based on the patient's 1DG and fasting glucose measured from a preceding day and nutrition data collected based on the report of food consumed by the patient during the current day. The model 815 iteratively repeats the process described daily over the course of a time period to predict the daily 1DG progression of the patient for each day of the time period. When implemented in accordance with the description above, the 1DG model generates predictions with a MAE of less than 6.0 over a 14-day sequence. Based on these patient-specific predictions, coaches or medical professionals are enabled to understand the impact of a particular nutrients or food items on a patient's metabolic state and may modify a treatment recommendation based on that understanding. Accordingly, a coach may collaborate with the digital twin module 350 to create a patient-specific nutrition recommendation that significantly reduces a patient's blood glucose levels to treat their diabetes or maintains the patient's reduced blood glucose levels. The 1DG model 815 may also be used to improve a patient's overall experience using the patient health management platform. For example, if a continuous glucose monitor becomes defective or a patient opts out of wearing a glucose monitor, the trained 1DG model 815 may be implemented as an effective replacement for the continuous glucose monitor by generating accurate predictions of a patient's 1DG over long time periods.

Because the 1DG model 815 generates iterative daily predictions based, in part, on the predictions from the previous day, deviations between the predictions of the glucose levels and the true glucose levels from preceding days are compounded into the current day's prediction. To address these compounding deviations, the short-term prediction module 810 implements the corrective model 820, which adjusts each prediction of the 1DG model 815 towards the patient's true glucose levels. The corrective model 820 is trained during the initialization period when the patient wears a physical CGM to measure their true glucose levels. The training dataset upon which the corrective model 820 is trained includes, for each day, the patient's true glucose level and a record of additional biosignal inputs recorded during that day, for example carbs consumed that day and calories consumed that day. Accordingly, the corrective module 820 is trained to predict a patient's true 1DG on a given day based on biosignal inputs that the 1DG model 815 may not consider.

In one specific embodiment, the corrective model 820 implements gradient boosting techniques (e.g., gradient boosted decision trees from the CatBoost library) to process the recursively generated predictions of the 1DG model 815 and a selection of important nutritional inputs including, but not limited to, daily carbohydrate consumption, calorie consumption, averaged glycemic index, net carbohydrate, and glycemic load to adjusted 1DG predictions.

For a period of time, the short-term prediction module 810 separates the predictions generated by the 1DG model 815 describing the patient's 1DG progression over the time period into shorter sequences of days of the time period. For each sequence of days leading up to a current day, the predictions generated by the 1DG model 815 and the additional curated set of nutrition data and metabolic data (e.g., the patient's true 1DG and metabolic state from the preceding day) are input to the trained corrective model 820 and the corrective model 820 generates an adjusted 1DG prediction for the current day that more closely resembles the patient's true 1DG on the current day. During training, the corrective model 820 compares a true glucose level measured on a given day be a patient's wearable sensor to the prediction generated by the 1DG model 815 to determine a deviation between the true glucose level and the predicted glucose level. The corrective model 820 identifies how particular nutrition and metabolic features impact the deviation between the predicted and true glucose levels to identify correlations between the deviations and the presence or values of the curated nutrition factors. When implemented, the corrective model 820 adjusts the prediction generated by the 1DG model 815 by anticipating the deviation between the prediction and the patient's true glucose level given the presence and values of one or more nutritional or metabolic features of the curated set discussed above.

The implementation of the corrective model 820 enables the short-term prediction model 810 to eliminate the error accumulation phenomenon described above that is characteristic of recursive prediction models including the 1DG model 815. Accordingly, the combination of the corrective model 820 with the 1DG model 815 generates 1DG predictions with a high-level of accuracy based on data accumulated over a time period, for example a period of 8-weeks. In some embodiments, a patient need only record 70% of the food items they consume to enable the short-term prediction module 810 to generate predictions with high accuracy. When implemented, the combination of the 1DG model 815 and the corrective model 820 may achieve an accuracy (i.e., a mean absolute error) of less than 8.8 mg/dL over 56 days.

As a patient continues to adhere to treatment recommendations generated by the platform 130 over a longer time period, the frequency with which the patient records nutrition data and the consumption of food items may decrease. The glucose twin module 515 recognizes when the patient is recording food items or nutrition data at a decreased frequency and compares that frequency to a threshold frequency. The threshold frequency may be dynamically defined by a medical provider or a health coach based on the patient's metabolic state determined for the previous day and a previous time period. As examples, the threshold frequency may be a daily recording of nutrition data or a semi-weekly recording of nutrition data. In response to determining that the frequency at which the patient is recording nutrition data is below the threshold frequency, the glucose twin module 515 applies the trained machine-learned model(s) of the long-term production module 825 to process a combination of input biosignals. The long-term prediction module 825 generates a personalized prediction of a patient's current metabolic state as measured by their average blood glucose based on passively collected biological data (e.g., sensor data and lab test data) rather than actively recorded patient data (e.g., nutrition data, symptom data, medication data). In some embodiments, in response to determining that nutrition data for a current day or time period is unavailable, the glucose twin module 515 may also apply the trained machine-learned model(s) of the long-term production module 825 to generate a prediction of a patient's current metabolic state.

Compared to the short-term prediction module 810 which leverages the relationship between nutrition data and measurements of a patient's glucose levels, the long-term prediction module 825 may leverage the relationship between a patient's physical activity and their heart rate. In one embodiment, the patient's physical activity is characterized based on a number of steps that they have taken time over a time period. After exercise the heart rate of a healthy patient returns (or begins to return) to a normal resting heart rate quickly, whereas the heart rate of a patient with poor metabolism returns to a normal resting heart rate much slower. Accordingly, the long-term prediction module 825 evaluates a patient's current metabolic state by correlating a patient's heart rate patterns with an amount of time elapsed since exercise and a level of that exercise. For example, if a patient finishes an exercise, but 30 minutes later there is no change in their heart rate, the long-term prediction module 825 determines that the patient's metabolic state has deteriorated from an improved state or has not improved from its previous state.

In one embodiment, the long-term prediction module 825 implements two separate machine-learned models: a first sequential data model 830 trained based on sensor data that changes over time (hereafter referred to as “sequential features”) and a second static data model 835 trained based on data that is fixed over time (hereafter referred to as “static features”). Examples of sequential features include, but are not limited to, heart rate measurements, minute-by-minute step counts, patient medication information that changes over time, and time-based interaction features (e.g., time elapsed between consecutive inputs, time elapsed between inputs and labels, etc.). Similar to the description above of the 1DG model 815, sequential features may be divided into smaller subsets of data recorded over periodic intervals, for example 3-day sequences. Examples of static features include, but are not limited, the patient medication that is constant over time, lab test data, and features derived from the lab test data. Examples of static lab test features include, but are not limited to, “ast”, “redBloodCells”, “hemoglobin”, “glucose”, “insulin”, “protein”, “urea”, “potassium”, “creatinine”, “sodium”, “chloride”, “calcium”, “veinHba1c”, and “ketoacidosis”. Static features are measured or calculated based on the results of a patient's most recent lab tests. Static features measured or calculated based on the most recent lab test are carried forward for each day leading up to the patient's next lab test or set of lab tests. Each of the above static features of lab test data are described above with reference to Table 1. Examples of static features derived from lab test data include, but are not limited to, “homaIR”, “measured_homaIR”, “tghdlratio”, “tgglucindex”, “measured_tghdlratio”, “measured_tgglucindex”, “glucscore”, “ketscore”, “glucose_ketone_index”, “cpepcgm”, “hgp_score”. Each of the derived static features are further described below.

The derived feature “homaIR” represents the Homeostatic Model Assessment for Insulin Resistance, which is a measurement of the amount of insulin resistance a patient exhibits. The feature “homaIR” may be calculated based on lab test data derived from a bloodwork test, for example according to Equation (1):

$$homalIR = \frac{[ glucose in \frac{m mol}{L} ] x [ insulin in \frac{m mol}{L} ]}{22.5} \qquad Eq . ( 1 )$$

The derived feature “tghdlratio” represents a ratio of triglycerides to high density lipoproteins in the blood, which may be interpreted as a proxy for insulin resistance. The feature “tghdlratio” may be calculated, for example according to Equation (2):

$$tghdlratio = \frac{triglycerides in mg / dL}{HDL cholesterol in mg / dL} \qquad Eq . ( 2 )$$

The derived feature “tgglucindex” represents a result of applying a logarithmic function to a product of triglycerides and fasting glucose levels, which is a value related to insulin resistance. The feature “tgglucindex” may be calculated, for example according to Equation (3):

$$tgglucindex = log log ( triglycerides in \frac{mg}{dL} \times glucose in \frac{mg}{dL} ) \qquad Eq . ( 3 )$$

The feature “measured_homaIR” is a Boolean value indicating whether the “homaIR” value was measured for a particular day or if it was forward-filled from a previous measurement. Similarly, the features “measured_tghdlratio” and “measured_tgglucindex” are Boolean values indicating whether those metrics were measured on a particular day, or if they were forward-filled.

The derived feature “glucscore” is a score that digitally represents a patient's glucose level. In one embodiment, a “glucscore” of 0 represents a 3-day mean glucose level below 125 mg/dL. A “glucscore” of 1 represents a mean glucose level between 125 and 137 mg/dL. A “glucscore” of 2 represents a mean glucose level between 137 and 145 mg/dL. A “glucscore” of 3 represents a mean glucose level between 145 and 160 mg/dL. A “glucscore” of 4 represents a mean glucose level between 160 and 200 mg/dL. A “glucscore” of 5 represents a mean glucose level greater than 200 mg/dL. In other embodiments, the long-term prediction module 825 may define additional or fewer scores representing different or varying ranges of glucose levels to represent a patient's glucose levels at various granularities.

The derived feature “ketscore” is a score that digitally represents a patient's Beta-hydroxybutyrate (BHB) level. In one embodiment, a “ketscore” of 1 represents a BHB level less than 0.4 mM, a “ketscore” of 1 corresponds to a BHB level between 0.4 and 0.7 mM, a “ketscore” of 2 represents a BHB level between 0.7 and 1.2 mM, a “ketscore” of 3 corresponds to a BHB level between 1.2 and 2.0 mM, a “ketscore” of 4 corresponds to a BHB level between 2.0 and 3.0 mM, a “ketscore” of 5 corresponds to a BHB level between 3.0 and 4.0 mM, and a “ketscore” of 6 corresponds to a BHB level greater than 4.0 mM. In other embodiments, the long-term prediction module 825 may define additional or fewer scores representing different or varying ranges of glucose levels to represent a patient's glucose levels at various granularities.

The derived feature “cpepcgm” is a product of the glucose level in the blood (mg/dL) and the C-peptide level in the blood (ng/mL), which is representative of insulin resistance. C-peptide is a marker of endogenous insulin product and has a longer half-life than insulin itself but is insensitive to exogenous insulin. The measurement of the glucose level in the blood may be an average of the glucose reported by a continuous glucose monitor over the 2 hours prior to a blood draw from which the C-peptide level is measured. The feature “cpepcgm” may be calculated, for example according to Equation (4):

$$cpepcgm = [ mean glucose in \frac{mg}{dL} ] \times [ C - peptide conentration in \frac{ng}{mL} ] \qquad Eq . ( 4 )$$

The derived feature “hgp_score” represents the incremental Area Under the Curve (AUC) of a glucose spike (h*mg/dL), which is measured at least five hours after the patient's last meal and while the patient is sleeping. Accordingly, any glucose spikes can be attributed to hepatic glucose production rather than digested food.

The feature “glucose_ketone_index” is a comparison of the “glucscore” described above and the “ketscore” described above. The “glucose_ketone_index” models the relationship between a patient's glucose levels and their BHB levels. In a healthy patient, BHB levels and glucose levels are inversely related. Accordingly, the values for the “glucose_ketone_index” can be determined based on the “glucscore” and the “ketscore” according to Table 2.

TABLE 2
Illustrative Interpretation of “Glucose_Ketone_Index”

ketscore

| | | 0 | 1 | 2 | 3 | 4 | 5 | 6 |
|---|---|---|---|---|---|---|---|---|
| glucscore | 0 | 5 | 5 | 5 | 5 | 2 | 2 | 5 |
| | 1 | 5 | 5 | 5 | 5 | 2 | 2 | 2 |
| | 2 | 4 | 4 | 2 | 2 | 2 | 2 | 1 |
| | 3 | 3 | 3 | 2 | 2 | 2 | 1 | 1 |
| | 4 | 3 | 3 | 1 | 1 | 1 | 1 | 1 |
| | 5 | 1 | 1 | 1 | 1 | 1 | 1 | 1 |

The label “hba1c” represents a patient's estimated hbA1c, also referred to as the “eA1c.” eA1c is estimation of a patient's long-term blood sugar recommended by the American Diabetes Association (ADA) and commonly used across the medical community. eA1c may be calculated, for example according to Equation (5):

$$eA 1 c ( \% ) = 46.7 + \frac{mean glocuse in mg / dL}{28.7} \qquad Eq . ( 5 )$$

In an alternative implementation, the static data model 835 may implement the label “effectiveGmi,” which refers to a patient's Glucose Management Indicator (GMI), in place of an eA1c estimation. Accordingly, GMI is an alternate method for estimating long-term blood sugar from CGM data. GMI may be calculated, for example according to Equation (6):

$$GMI ( \% ) = 3.31 + ( 0.2392 \times [ mean glucose in \frac{mg}{dL} ] ) \qquad Eq . ( 6 )$$

In one embodiment, the sequential data model 830 implements a long-short term memory recurrent neural network (LSTM) with GRU cells (e.g., LSTM and GRU layers from the Tensorflow library). LSTM and GRU cells are variants of recurrent neural networks that allow for the training of models capable of learning over long sequences of time variant data. As described with reference to FIG. 6, a baseline LSTM is trained using a training dataset of data from a population of patients. Next, a personalized LSTM is generated by retraining the baseline LSTM model using a training dataset of data collected for a particular patient. Accordingly, the weights of the personalized LSTM model are initialized using the trained weights from the baseline LSTM, such that the personalized LSTM represents a variation of the baseline LSTM that is fine-tuned based on the patient's own data. Each GRU cell in the personalized LSTM model represents a subset of the sequential features (e.g., sequential features recorded during a three-day interval), such that the LSTM model iteratively processes sequential features recorded over a time period to output a sequential eA1c prediction.

The static data model 835 implements a neural network with Dense layers (e.g., a Dense neural network from the Tensorflow library). The static data model 835 receives, as inputs, static features measured or derived from a patient's most recent lab tests to output a static eA1c prediction. As described with reference to FIG. 6, a baseline Dense neural network is trained using a training dataset of data from a population of patients. Next, a personalized Dense neural network is generated by retraining the baseline Dense neural network using a training dataset of exclusively the patient's own data. Accordingly, the weights of the personalized Dense neural network are initialized using the trained weights from the Dense neural network, such that the personalized Dense neural network represents a variation of the baseline Dense neural network that is fine-tuned based on the patient's own data.

In one embodiment, both the sequential data model 830 and the static data model 835 are trained based on two thirds of a patient's personalized training dataset and evaluated based on the remaining third of the patient's personalized training dataset.

The A1c estimation module 840 concatenates the sequential A1c estimation output by the sequential data model 830 with the static A1c estimation output static data model 835 to generate an aggregate prediction of a patient's rolling blood glucose average, for example a 10-day rolling blood glucose average. The concatenation performed by the A1c estimation module 840 is further described with reference to FIG. 8D.

When implemented in accordance with the description above, the A1c estimation module 840 generates an aggregate prediction with Mean Absolute Error (MAE) of 0.44. Additionally, as described above, the long-term prediction module 825 is a time-independent combination of machine-learned models that may operate indefinitely throughout the future. When implemented in accordance with the description above, the aggregate prediction may be accurate for over 18 months from a patient's last physical CGM reading with a MAE of 0.44.

FIG. 8B is an illustration of the process for determining a short-term prediction of glucose levels for a patient, according to one embodiment. The glucose twin module 515 accesses 851 biosignal data recorded for a patient over a sequence of days. For a first day in the sequence, the glucose twin module 515 predicts 852 a patient's glucose levels based on the patient's current metabolic state, biosignal data recorded during the first day, and food consumed by the patient during the first day. For each subsequent day in the sequence of days, the glucose twin module 515 recursively predicts 853 the patient's glucose levels based on the food consumed by the patient on that day, the biosignal data recorded during that day, and the glucose levels predicted from the previous day. Accordingly, a prediction of a patient's most recent glucose levels is influenced by the food most recently consumed by the patient and by their historical glucose levels. When recursively generating predictions of the patient's glucose levels, the glucose twin module 515 divides the sequence of days into smaller subsets of days and generates glucose level predictions for each subset. The glucose twin module 515 additionally inputs 854 the predicted glucose level for each subset and an additional set of curated nutritional data to a corrective model to determine an adjusted prediction of the patient's glucose levels that more closely resembles the true glucose levels for a patient.

FIG. 8C is an illustration of the process for determining a long-term prediction of glucose levels for a patient, according to one embodiment. The glucose twin module 515 accesses biosignal data recorded for the patient over a time period. From the accessed biosignal data, the glucose twin module 515 determines 861 one or more sequential features of the patient's metabolic state and determines 862 one or more static features of the patient's metabolic state. Sequential features, as described with reference to FIG. 8A, represent biosignals with values that dynamically change over time and static features represent biosignals with values that are constant over time. The glucose twin module 515 divides 863 the set of sequential features into subsets of sequential features recorded at periodic intervals along the time period, for example three-day intervals. The glucose twin module 515 determines 864 a sequential prediction of the patient's glucose levels by recursively inputting the sequential features from each interval to a first machine-learned model. Additionally, the glucose twin module 515 determines 865 a prediction of the patient's glucose levels at the conclusion of the time period by inputting the static features to a second machine-learned model. The first machine-learned model may be a recursive neural network, for example an LSTM recurrent neural network, and the second machine-learned model may be a conventional neural network, for example a Dense neural network. The glucose twin module 651 determines 866 an aggregate estimate of the patient's A1c for a rolling 10-day blood glucose average by concatenating the prediction output by the first model with the prediction output by the second model.

FIG. 8D is an illustration of a flowchart for concatenating an A1c prediction generated based on sequential features with an A1c prediction generated based on static features, according to one embodiment. As discussed above, the long-term prediction module 825 implements a deep neural network 870 with multiple, jointly-trained input channels to generate predictions of a patient's glucose levels based on a combination of sequential and static input features. The multiple channels enable the long-term prediction model to process particular types of input feature (e.g., a sequential input feature or a static input feature).

A first input channel 871 of the deep neural network generates a sequential A1c estimation by passing sequential data through a series of recurrent neural network layers, for examples LSTM cells 872. As discussed above, LSTM cells 872 are specialized components capable of modeling complex sequential dynamics present in biometric data. This first input channel 871 may be implemented in a flexible fashion that enables the sequential data model 830 to accommodate input sequences of varying lengths. A second input channel 873 of the deep neural network generates a static A1c estimation by passing static data through a stack of standard dense layers 874 of the neural network. In one embodiment, each dense layer 874 comprises 64 neurons.

The first input channel 871 and the second input channel 873 converge at a concatenation layer 875 of the deep neural network 870, where the A1c estimation module 840 combines the signals from the two input channels into an aggregate estimate of a patient's A1C. The A1c estimation module 840 passes the aggregated estimate through a series of dense, fully connected layers to determine the combination of information gained from each of the two input channels that will yield the most or most relevant insight into a patient's metabolic state.

IV.E Patient-Specific Recommendations

The recommendation module 360 may include a combination of rule-based artificial intelligence techniques representing codified medical knowledge from established medical practice (e.g., American Diabetes Association guidelines, research literature, and insights gained from past medical treatments). The recommendation module 360 applies the codified knowledge in an automated manner to recommend treatments for new patients using the patient health management platform 130.

The platform 130 additionally categorizes patients into a cohort with other patients with similar metabolic profiles. The recommendation module 360 applies a system of rule to assign patients with a similar metabolic profile to the same cohort. The recommendation module 360 then tailors a specific treatment recommendation (i.e., a combination of nutrition and medication regimens) for the metabolic profiles of patients in each cohort. In some implementations, the recommendation module 360 generates a representative metabolic profile for each cohort based on an average of the metabolic profiles for each patient in cohort or an aggregate of the metabolic profiles for each patient in cohort. The rule-based intelligence applied to categorize patients in cohorts is based on biosignals characterizing a patient's metabolic state or general health, for example biosignals recorded by wearable sensors or measured using lab tests. Specific examples of such cohorting rules include, but are not limited to, BMI, 5-day average blood glucose (“5DG”), 5-day average of grams of net carbs eaten per day (“5 dgnc”), 5-day average of the number of >50 mg/dL blood glucose spikes per day (“5 dspike”), ketone levels, and whether the patient is taking medications like glimepiride.

### V. Additional Considerations

It is to be understood that the figures and descriptions of the present disclosure have been simplified to illustrate elements that are relevant for a clear understanding of the present disclosure, while eliminating, for the purpose of clarity, many other elements found in a typical system. Those of ordinary skill in the art may recognize that other elements and/or steps are desirable and/or required in implementing the present disclosure. However, because such elements and steps are well known in the art, and because they do not facilitate a better understanding of the present disclosure, a discussion of such elements and steps is not provided herein. The disclosure herein is directed to all such variations and modifications to such elements and methods known to those skilled in the art.

Some portions of the above description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like.

Any of the steps, operations, or processes described herein may be performed or implemented with one or more hardware or software modules, alone or in combination with other devices. In one embodiment, a software module is implemented with a computer program product including a computer-readable non-transitory medium containing computer program code, which can be executed by a computer processor for performing any or all of the steps, operations, or processes described.

Embodiments of the invention may also relate to a product that is produced by a computing process described herein. Such a product may include information resulting from a computing process, where the information is stored on a non-transitory, tangible computer readable storage medium and may include any embodiment of a computer program product or other data combination described herein.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

While particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed is:

1. A method for predicting a blood glucose level of a patient, the method comprising:
accessing, by a health management platform from a data store, a metabolic state of the patient and plurality of biosignals recorded for the patient during a time period, the plurality of biosignals comprising one or more of: 1) nutrition data of food items consumed by the patient during a current day, 2) a fasting glucose level predicted for a preceding day, and 3) sensor data and lab test data recorded during the time period;
encoding the plurality of biosignals into a vector representation;
applying a patient-specific metabolic model to the vector representation to generate a prediction of a glucose level for the patient during the current day, wherein the patient-specific metabolic model is trained based on a training dataset of true glucose measurements recorded for the patient during an initialization period and historical biosignals recorded for the patient that contributed to each true glucose measurement;
for each day of the time period, applying a patient-specific corrective model to the predicted glucose level of the patient to generate an updated prediction of the glucose level of the patient, wherein the patient-specific corrective model generates the updated prediction by adjusting the glucose level predicted by the patient-specific metabolic model towards a true glucose level of the patient on the current day; and
generating, for display on a mobile device, a notification describing the updated prediction of the glucose level of the patient.

2. The method of claim 1, wherein training the patient-specific metabolic model comprises:
identifying, from a population of patients, one or more sub-populations of patients with similar metabolic states to the metabolic state of the patient;
training a baseline metabolic model to generate a glucose level prediction based on a training dataset of sensor data and lab test data collected for the one or more sub-populations;
generating and iteratively training the patient-specific metabolic model by applying the baseline metabolic model to the training dataset of true glucose measurements recorded for the patient and historical biosignals that contributed to each true glucose measurement.

3. The method of claim 1, wherein applying the predicted glucose level of the patient to the patient-specific corrective model to generate the updated prediction comprises:
determining a deviation between the predicted glucose level and the true glucose level of the patient on the current day based on a subset of nutrition data and biosignals collected by wearable sensors on the current day; and
generating the updated prediction by adjusting the predicted glucose level based on the deviation.

4. The method of claim 3, further comprising:
for each day of the initialization period,
measuring, by a wearable sensor, sensor data describing a true glucose level of the patient;
accessing the prediction of the glucose level for the patient generated by the patient-specific metabolic model;
determining a deviation between the predicted glucose level and the measured true glucose level based on a comparison; and
generating the subset of nutrition data and biosignals by identifying nutrition data and biosignals correlated with the deviation between the measured true glucose level and the predicted glucose level.

5. The method of claim 1, further comprising:
determining a frequency with which the patient reports nutrition data during the time period;
responsive to determining that the determined frequency is less than a threshold frequency, accessing additional biosignals recorded for the patient during the time period, the additional biosignals comprising at least one of physical activity data and heart rate data collected during the time period by wearable sensors worn by the patient;
encoding the plurality of biosignals and the additional biosignals into a second vector representation;
applying a long-term metabolic model to the second vector representation to generate a long-term glucose level prediction of the patient during the time period, wherein the long-term model is trained based on a long-term training dataset of true glucose measurements recorded for the patient during the initialization period and historical biosignals recorded for the patient that contributed to each true glucose measurement; and
generating, for display on the mobile device, a notification describing the long-term glucose level prediction of the patient.

6. The method of claim 5, wherein training the long-term metabolic model comprises:
generating and iteratively training the patient-specific metabolic model by applying a baseline metabolic model to the long-term training dataset of true glucose measurements recorded for the patient and historical biosignals that contributed to each true glucose measurement.

7. The method of claim 5, wherein the second vector representation identifies static features and sequential features, the long-term prediction model further trained to:
input static features to a static feature submodel to generate a static glucose level prediction for the time period; and
input sequential features to a sequential feature submodel to generate a sequential glucose level prediction for the time period; and
concatenate the static glucose level prediction with the sequential glucose level prediction to generate an aggregate prediction of a rolling glucose level of the patient during the time period.

8. The method of claim 5, wherein the long-term prediction of the glucose level is an estimation of a hemoglobin A1c level of the patient during the time period.

9. The method of claim 5, further comprising:
modifying a digital representation of the metabolic state of the patient based on the long-term glucose level prediction of the patient.

10. A non-transitory computer readable medium storing instructions for predicting a blood glucose level of a patient encoded thereon that, when executed by a processor, cause the processor to:
access, by a health management platform from a data store, a metabolic state of the patient and plurality of biosignals recorded for the patient during a time period, the plurality of biosignals comprising one or more of: 1) nutrition data of food items consumed by the patient during a current day, 2) a fasting glucose level predicted for a preceding day, and 3) sensor data and lab test data recorded during the time period;
encode the plurality of biosignals into a vector representation;
apply a patient-specific metabolic model to the vector representation to generate a prediction of a glucose level for the patient during the current day, wherein the patient-specific metabolic model is trained based on a training dataset of true glucose measurements recorded for the patient during an initialization period and historical biosignals recorded for the patient that contributed to each true glucose measurement;
for each day of the time period, apply a patient-specific corrective model to the predicted glucose level of the patient to generate an updated prediction of the glucose level of the patient, wherein the patient-specific corrective model generates the updated prediction by adjusting the glucose level predicted by the patient-specific metabolic model towards a true glucose level of the patient on the current day; and
generate, for display on a mobile device, a notification describing the updated prediction of the glucose level of the patient.

11. The non-transitory computer readable medium of claim 10, wherein the instructions for training the patient-specific metabolic model further cause the processor to:
identify, from a population of patients, one or more sub-populations of patients with similar metabolic states to the metabolic state of the patient;
train a baseline metabolic model to generate a glucose level prediction based on a training dataset of sensor data and lab test data collected for the one or more sub-populations;
generate and iteratively training the patient-specific metabolic model by applying the baseline metabolic model to the training dataset of true glucose measurements recorded for the patient and historical biosignals that contributed to each true glucose measurement.

12. The non-transitory computer readable medium of claim 10, wherein the instructions for applying the predicted glucose level of the patient to the second patient-specific model to generate the updated prediction further cause the processor to:
determine a deviation between the predicted glucose level and the true glucose level of the patient on the current day based on a subset of nutrition data and biosignals collected by wearable sensors on the current day; and
generate the updated prediction by adjusting the predicted glucose level based on the deviation.

13. The non-transitory computer readable medium of claim 12, further comprising instructions that cause the processor to:
for each day of the initialization period,
measure, by a wearable sensor, sensor data describing a true glucose level of the patient;
access the prediction of the glucose level for the patient generated by the patient-specific metabolic model;
determine a deviation between the predicted glucose level and the measured true glucose level based on a comparison; and
generate the subset of nutrition data and biosignals by identifying nutrition data and biosignals correlated with the deviation between the measured true glucose level and the predicted glucose level.

14. The non-transitory computer readable medium of claim 10, further comprising instructions that cause the processor to:
determine a frequency with which the patient reports nutrition data during the time period;
responsive to determining that the determined frequency is less than a threshold frequency, access additional biosignals recorded for the patient during the time period, the additional biosignals comprising at least one of physical activity data and heart rate data collected during the time period by wearable sensors worn by the patient;
encode the plurality of biosignals and the additional biosignals into a second vector representation;
apply a long-term metabolic model to the second vector representation to generate a long-term glucose level prediction of the patient during the time period, wherein the long-term model is trained based on a long-term training dataset of true glucose measurements recorded for the patient during the initialization period and historical biosignals recorded for the patient that contributed to each true glucose measurement; and
generate, for display on the mobile device, a notification describing the long-term glucose level prediction of the patient.

15. The non-transitory computer readable medium of claim 14, wherein the instructions for training the long-term metabolic model further cause the processor to:
generate and iteratively training the patient-specific metabolic model by applying a baseline metabolic model to the long-term training dataset of true glucose measurements recorded for the patient and historical biosignals that contributed to each true glucose measurement.

16. The non-transitory computer readable medium of claim 14, wherein the second vector representation identifies static features and sequential features, the long-term prediction model further trained to:
input static features to a static feature submodel to generate a static glucose level prediction for the time period; and
input sequential features to a sequential feature submodel to generate a sequential glucose level prediction for the time period; and
concatenate the static glucose level prediction with the sequential glucose level prediction to generate an aggregate prediction of a rolling glucose level of the patient during the time period.

17. The non-transitory computer readable medium of claim 14, wherein the long-term prediction of the glucose level is an estimation of a hemoglobin A1c level of the patient during the time period.

18. The non-transitory computer readable medium of claim 14, further comprising instructions that cause the processor to:
modify a digital representation of the metabolic state of the patient based on long-term glucose level prediction of the patient.

19. A method for predicting a blood glucose level of a patient, the method comprising:
accessing, by a health management platform from a data store, a metabolic state of the patient and plurality of biosignals recorded for the patient during a time period, the plurality of biosignals comprising one or more of: 1) at least one of physical activity data and heart rate data collected during the time period by one or more wearable sensors worn by the patient, 2) a fasting glucose level predicted for a preceding day, and 3) lab test data recorded during the time period;
identifying, from the accessed biosignals, a first subset of sequential biosignals with values that are dynamic at daily intervals and a second subset of static biosignals with values that are static at daily intervals;
encoding the first subset of sequential biosignals into a sequential vector representation and the second subset of static biosignals into a static vector representation;
applying a first patient-specific metabolic model to the sequential vector representation to determine an estimation of blood glucose for the patient during the time period based on the first subset of sequential biosignals, wherein the first patient-specific metabolic model is trained based on a training dataset of true glucose measurements recorded during an initialization period and sequential biosignals that contributed to each true glucose level;
applying a second patient-specific metabolic model to the static vector representation to determine an estimation of blood glucose for the patient during the time period based on the second subset of static biosignals, wherein the second patient-specific metabolic model is trained based on a training dataset of true glucose levels and historical static biosignals that contributed to each true glucose level;
determining an aggregate blood glucose estimate for the patient based on the estimation determined by the first patient-specific metabolic model combined with the estimation determined by the second patient-specific metabolic model; and
generating, for display on a mobile device, a notification describing the aggregate blood glucose estimate for the patient.

20. The method of claim 19, wherein training the first patient-specific metabolic model comprises:
generating and iteratively training the first patient-specific metabolic model using the training dataset of true glucose measurements recorded during an initialization period and sequential biosignals that contributed to each true glucose level.

21. The method of claim 19, wherein training the second patient-specific metabolic model comprises:
generating and iteratively training the second patient-specific metabolic model using the training dataset of true glucose measurements recorded during an initialization period and static biosignals that contributed to each true glucose level.

22. The method claim 19, wherein the long-term prediction of the glucose level is an estimation of the hemoglobin A1c level of the patient during the time period.

23. The method of claim 19, further comprising:
modifying a digital representation of the metabolic state of the patient based on the aggregate blood glucose estimate for the patient.
